SEQUENCE LISTING (9 sequences)

SEQ ID NO: 1 - protein, 679 aa, Homo sapiens
MISASRAAAARLVGAAASRGPTAARHQDSWNGLSHEAFRLVSRRDYASEAIKGAVVGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 2 - DNA, 2850 bp, Homo sapiens
tcaccccacgtggttggaggtttccagaagcgctgccgccaccgcatcgcgcagctctttgccgtcggagcgcttgtttgctgcctcgtactcctccatttatccgccatgataagtgccagccgagctgcagcagcccgtctcgtgggcgccgcagcctcccggggccctacggccgcccgccaccaggatagctggaatggccttagtcatgaggcttttagacttgtttcaaggcgggattatgcatcagaagcaatcaagggagcagttgttggtattgatttgggtactaccaactcctgcgtggcagttatggaaggtaaacaagcaaaggtgctggagaatgccgaaggtgccagaaccaccccttcagttgtggcctttacagcagatggtgagcgacttgttggaatgccggccaagcgacaggctgtcaccaacccaaacaatacattttatgctaccaagcgtctcattggccggcgatatgatgatcctgaagtacagaaagacattaaaaatgttccctttaaaattgtccgtgcctccaatggtgatgcctgggttgaggctcatgggaaattgtattctccgagtcagattggagcatttgtgttgatgaagatgaaagagactgcagaaaattacttggggcacacagcaaaaaatgctgtgatcacagtcccagcttatttcaatgactcgcagagacaggccactaaagatgctggccagatatctggactgaatgtgcttcgggtgattaatgagcccacagctgctgctcttgcctatggtctagacaaatcagaagacaaagtcattgctgtatatgatttaggtggtggaacttttgatatttctatcctggaaattcagaaaggagtatttgaggtgaaatccacaaatggggataccttcttaggtggggaagactttgaccaggccttgctacggcacattgtgaaggagttcaagagagagacaggggttgatttgactaaagacaacatggcacttcagagggtacgggaagctgctgaaaaggctaaatgtgaactctcctcatctgtgcagactgacatcaatttgccctatcttacaatggattcttctggacccaagcatttgaatatgaagttgacccgtgctcaatttgaagggattgtcactgatctaatcagaaggactatcgctccatgccaaaaagctatgcaagatgcagaagtcagcaagagtgacataggagaagtgattcttgtgggtggcatgactaggatgcccaaggttcagcagactgtacaggatctttttggcagagccccaagtaaagctgtcaatcctgatgaggctgtggccattggagctgccattcagggaggtgtgttggccggcgatgtcacggatgtgctgctccttgatgtcactcccctgtctctgggtattgaaactctaggaggtgtctttaccaaacttattaataggaataccactattccaaccaagaagagccaggtattctctactgccgctgatggtcaaacgcaagtggaaattaaagtgtgtcagggtgaaagagagatggctggagacaacaaactccttggacagtttactttgattggaattccaccagcccctcgtggagttcctcagattgaagttacatttgacattgatgccaatgggatagtacatgtttctgctaaagataaaggcacaggacgtgagcagcagattgtaatccagtcttctggtggattaagcaaagatgatattgaaaatatggttaaaaatgcagagaaatatgctgaagaagaccggcgaaagaaggaacgagttgaagcagttaatatggctgaaggaatcattcacgacacagaaaccaagatggaagaattcaaggaccaattacctgctgatgagtgcaacaagctgaaagaagagatttccaaaatgagggagctcctggctagaaaagacagcgaaacaggagaaaatattagacaggcagcatcctctcttcagcaggcatcactgaagctgttcgaaatggcatacaaaaagatggcatctgagcgagaaggctctggaagttctggcactggggaacaaaaggaagatcaaaaggaggaaaaacagtaataatagcagaaattttgaagccagaaggacaacatatgaagcttaggagtgaagagacttcctgagcagaaatgggcgaacttcagtctttttactgtgtttttgcagtattctatatataatttccttaatttgtaaatttagtgaccattagctagtgatcatttaatggacagtgattctaacagtataaagttcacaatattctatgtccctagcctgtcatttttcagctgcatgtaaaaggaggtaggatgaattgatcattataaagatttaactattttatgctgaagtgaccatattttcaaggggtgaaaccatctcgcacacagcaatgaaggtagtcatccatagacttgaaatgagaccacatatggggatgagatccttctagttagcctagtactgctgtactggcctgtatgtacatggggtccttcaactgaggccttgcaagtcaagctggctgtgccatgtttgtagatggggcagaggaatctagaacaatgggaaacttagctatttatattaggtacagctattaaaacaaggtaggaatgaggctagacctttaacttccctaaggcatacttttctagctaccttctgccctgtgtctggcacctacatccttgatgattgttctcttacccattctggaatttttttttttttaaataaatacagaaagcatcttga

SEQ ID NO: 3 - protein, 381 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 4 - protein, 392 aa, Homo sapiens [sequence omitted]

SEQ ID NO: 5 - DNA, 3371 bp, Homo sapiens
gctcgggccacgcccacctgtcctgcagcactggatgctttgtgagttggggattgttgcgtcccatatctggacccagaagggacttccctgctcggctggctctcggtttctctgctttcctccggagaaataacagcgtcttccgcgccgcgcatggagcctcccggccgccgcgagtgtccctttccttcctggcgctttcctgggttgcttctggcggccatggtgttgctgctgtactccttctccgatgcctgtgaggagccaccaacatttgaagctatggagctcattggtaaaccaaaaccctactatgagattggtgaacgagtagattataagtgtaaaaaaggatacttctatatacctcctcttgccacccatactatttgtgatcggaatcatacatggctacctgtctcagatgacgcctgttatagagaaacatgtccatatatacgggatcctttaaatggccaagcagtccctgcaaatgggacttacgagtttggttatcagatgcactttatttgtaatgagggttattacttaattggtgaagaaattctatattgtgaacttaaaggatcagtagcaatttggagcggtaagcccccaatatgtgaaaaggttttgtgtacaccacctccaaaaataaaaaatggaaaacacacctttagtgaagtagaagtatttgagtatcttgatgcagtaacttatagttgtgatcctgcacctggaccagatccattttcacttattggagagagcacgatttattgtggtgacaattcagtgtggagtcgtgctgctccagagtgtaaagtggtcaaatgtcgatttccagtagtcgaaaatggaaaacagatatcaggatttggaaaaaaattttactacaaagcaacagttatgtttgaatgcgataagggtttttacctcgatggcagcgacacaattgtctgtgacagtaacagtacttgggatcccccagttccaaagtgtcttaaagtgctgcctccatctagtacaaaacctccagctttgagtcattcagtgtcgacttcttccactacaaaatctccagcgtccagtgcctcaggtcctaggcctacttacaagcctccagtctcaaattatccaggatatcctaaacctgaggaaggaatacttgacagtttggatgtttgggtcattgctgtgattgttattgccatagttgttggagttgcagtaatttgtgttgtcccgtacagatatcttcaaaggaggaagaagaaaggcacatacctaactgatgagacccacagagaagtaaaatttacttctctctgagaaggagagatgagagaaaggtttgcttttatcattaaaaggaaagcagatggtggagctgaatatgccacttaccagactaaatcaaccactccagcagagcagagaggctgaatagattccacaacctggtttgccagttcatcttttgactctattaaaatcttcaatagttgttattctgtagtttcactctcatgagtgcaactgtggcttagctaatattgcaatgtggcttgaatgtaggtagcatcctttgatgcttctttgaaacttgtatgaatttgggtatgaacagattgcctgctttcccttaaataacacttagatttattggaccagtcagcacagcatgcctggttgtattaaagcagggatatgctgtattttataaaattggcaaaattagagaaatatagttcacaatgaaattatattttctttgtaaagaaagtggcttgaaatcttttttgttcaaagattaatgccaactcttaagattattctttcaccaactatagaatgtattttatatatcgttcattgtaaaaagcccttaaaaatatgtgtatactactttggctcttgtgcataaaaacaagaacactgaaaattgggaatatgcacaaacttggcttctttaaccaagaatattattggaaaattctctaaaagttaatagggtaaattctctattttttgtaatgtgttcggtgatttcagaaagctagaaagtgtatgtgtggcatttgttttcactttttaaaacatccctaactgatcgaatatatcagtaatttcagaatcagatgcatcctttcataagaagtgagaggactctgacagccataacaggagtgccacttcatggtgcgaagtgaacactgtagtcttgttgttttcccaaagagaactccgtatgttctcttaggttgagtaacccactctgaattctggttacatgtgtttttctctccctccttaaataaagagaggggttaaacatgccctctaaaagtaggtggttttgaagagaataaattcatcagataacctcaagtcacatgagaatcttagtccatttacattgccttggctagtaaaagccatctatgtatatgtcttacctcatctcctaaaaggcagagtacaaagtaagccatgtatctcaggaaggtaacttcattttgtctatttgctgttgattgtaccaagggatggaagaagtaaatatagctcaggtagcactttatactcaggcagatctcagccctctactgagtcccttagccaagcagtttctttcaaagaagccagcaggcgaaaagcagggactgccactgcatttcatatcacactgttaaaagttgtgttttgaaattttatgtttagttgcacaaattgggccaaagaaacattgccttgaggaagatatgattggaaaatcaagagtgtagaagaataaatactgttttactgtccaaagacatgtttatagtgctctgtaaatgttcctttcctttgtagtctctggcaagatgctttaggaagataaaagtttgaggagaacaaacaggaattctgaattaagcacagagttgaagtttatacccgtttcacatgcttttcaagaatgtcgcaattactaagaagcagataatggtgttttttagaaacctaattgaagtatattcaaccaaatactttaatgtataaaataaatattatacaatatacttgtatagcagtttctgcttcacatttgattttttcaaatttaatatttatattagagatctatatatgtataaatatgtattttgtcaaatttgttacttaaatatatagagaccagttttctctggaagtttgtttaaatgacagaagcgtatatgaattcaagaaaatttaagctgcaaaaatgtatttgctataaaatgagaagtctcactgatagaggttctttattgctcattttttaaaaaatggactcttgaaatctgttaaaataaaattgtacatttggagatgtttca

SEQ ID NO: 6 - protein, 128 aa, Homo sapiens
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 7 - DNA, 7633 bp, Homo sapiens
gggccggggggcggagccttgcgggctggagcgaaagaatgcgggggctgagcgcagaagcggctcgaggctggaagaggatcttgggcgccgccaggttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcggggggttggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaaaaaaaaaaaaaaaaaa

SEQ ID NO: 8 - DNA, 2094 bp, Homo sapiens
gcttgttccctgtcctctggccctttgcaaataaatgccttaccagacctgccctgccaccccactcgcagccacccagcaagagcagcatgtcagcctgccggagctttgcagttgcaatctgcattttagaaataagcatcctcacagcacagtacacgaccagttatgacccagagctaacagaaagcagtggctctgcatcacacatagactgcagaatgagcccctggagtgaatggtcacaatgcgatccttgtctcagacaaatgtttcgttcaagaagcattgaggtctttggacaatttaatgggaaaagatgcaccgacgctgtgggagacagacgacagtgtgtgcccacagagccctgtgaggatgctgaggatgactgcggaaatgactttcaatgcagtacaggcagatgcataaagatgcgacttcggtgtaatggtgacaatgactgcggagacttttcagatgaggatgattgtgaaagtgagccccgtcccccctgcagagacagagtggtagaagagtctgagctggcacgaacagcaggctatgggatcaacattttagggatggatcccctaagcacaccttttgacaatgagttctacaatggactctgtaaccgggatcgggatggaaacactctgacatactaccgaagaccttggaacgtggcttctttgatctatgaaaccaaaggcgagaaaaatttcagaaccgaacattacgaagaacaaattgaagcatttaaaagtatcatccaagagaagacatcaaattttaatgcagctatatctctaaaatttacacccactgaaacaaataaagctgaacaatgttgtgaggaaacagcctcctcaatttctttacatggcaagggtagttttcggttttcatattccaaaaatgaaacttaccaactatttttgtcatattcttcaaagaaggaaaaaatgtttctgcatgtgaaaggagaaattcatctgggaagatttgtaatgagaaatcgcgatgttgtgctcacaacaacttttgtggatgatataaaagctttgccaactacctatgaaaagggagaatattttgcctttttggaaacctatggaactcactacagtagctctgggtctctaggaggactctatgaactaatatatgttttggataaagcttccatgaagcggaaaggtgttgaactaaaagacataaagagatgccttgggtatcatctggatgtatctctggctttctctgaaatctctgttggagctgaatttaataaagatgattgtgtaaagaggggagagggtagagctgtaaacatcaccagtgaaaacctcatagatgatgttgtttcactcataagaggtggaaccagaaaatatgcatttgaactgaaagaaaagcttctccgaggaaccgtgattgatgtgactgactttgtcaactgggcctcttccataaatgatgctcctgttctcattagtcaaaaactgtctcctatatataatctggttccagtgaaaatgaaaaatgcacacctaaagaaacaaaacttggaaagagccattgaagactatatcaatgaatttagtgtaagaaaatgccacacatgccaaaatggaggtacagtgattctaatggatggaaagtgtttgtgtgcctgcccattcaaatttgagggaattgcctgtgaaatcagtaaacaaaaaatttctgaaggattgccagccctagagttccccaatgaaaaatagagctgttggcttctctgagctccagtggaagaagaaaacactagtaccttcagatcctacccctgaagataatcttagctgccaagtaaatagcaacatgcttcatgaaaatcctaccaacctctgaagtctcttctctcttaggtctataattttttttttaatttttcttccttaaactcctgtgatgtttccattttttgttccctaatgagaagtcaacagtgaaatacgccagaactgctttatcccacggaaaatgccaatctcttctaaaaaaaaacaaaattaaactaaaaaaaaaaaaaaaaaaaaaaaaaaaaaa

SEQ ID NO: 9 - protein, 559 aa, Homo sapiens
MSACRSFAVAICILEISILTAQYTTSYDPELTESSGSASHIDCRMSPWSEWSQCDPCLRQMFRSRSIEVFGQFNGKRCTDAVGDRRQCVPTEPCEDAEDDCGNDFQCSTGRCIKMRLRCNGDNDCGDFSDEDDCESEPRPPCRDRVVEESELARTAGYGINILGMDPLSTPFDNEFYNGLCNRDRDGNTLTYYRRPWNVASLIYETKGEKNFRTEHYEEQIEAFKSIIQEKTSNFNAAISLKFTPTETNKAEQCCEETASSISLHGKGSFRFSYSKNETYQLFLSYSSKKEKMFLHVKGEIHLGRFVMRNRDVVLTTTFVDDIKALPTTYEKGEYFAFLETYGTHYSSSGSLGGLYELIYVLDKASMKRKGVELKDIKRCLGYHLDVSLAFSEISVGAEFNKDDCVKRGEGRAVNITSENLIDDVVSLIRGGTRKYAFELKEKLLRGTVIDVTDFVNWASSINDAPVLISQKLSPIYNLVPVKMKNAHLKKQNLERAIEDYINEFSVRKCHTCQNGGTVILMDGKCLCACPFKFEGIACEISKQKISEGLPALEFPNEK

(19) United States
(12) Patent Application Publication
(10) Pub. No.: US 2011/0059070 A1
(43) Pub. Date: Mar. 10, 2011

(54) Combination therapy for tumoral desease treatment

(75) Inventors: Zvi Fishelson, Tel-Aviv (IL); David Pilzer, Holon (IL)

(73) Assignee: Ramot At Tel Aviv University Ltd., Tel-Aviv (IL)

(21) Appl. No.: 12/310,790

(22) PCT Filed: Sep. 11, 2007
(86) PCT No.: PCT/IL2007/001132

§ 371 Date: Sep. 2, 2009

Related U.S. Application Data
(60) Provisional application No. 60/844,398, filed on Sep. 14, 2006.

(51) Int. Cl. A61K 39/395; G01N 33/53; A61P 35/00

(52) U.S. Cl. 424/133.1; 424/130.1; 435/7.21

(57) ABSTRACT

The invention provides a method of treating cancer by administering to a subject in need thereof, the subject being treated with an anti-cancer antibody therapy, a therapeutically effective amount of a delocalized lipophilic cation (DLC) compound such as MKT-077 which is capable of binding mortalin as an adjuvant for the anti-cancer antibody therapy. Also provided are pharmaceutical compositions and article of manufacturer for treating cancer which comprise the DLC compound as an adjuvant for anti-cancer antibody therapy.

### FIELD AND BACKGROUND OF THE INVENTION

The invention relates to pharmaceutical compositions and methods of treating cancer using a delocalized lipophilic cation (DLC) compound which sensitizes cells to anti-cancer antibody therapy, and more particularly, to the use of such a compound as an adjuvant for an anti-cancer antibody therapy.

The complement system plays a major role in natural resistance to bacterial and viral infections via membranolysis, leukocyte chemotaxis, opsonization, enhancement of phagocytosis and release of mediators of inflammation. The final membranolytic product is a protein complex composed of C5b, C6, C7 C8 and up to 18 copies of C9 which is referred to as the membrane attack complex (MAC) or the terminal complement complex. The MAC is known to induce (at high doses) plasma membrane damage, calcium ions influx and mitochondrial damage to target cells, leading to ATP depletion and necrotic type cell death. Additionally, under certain conditions the MAC is capable of inducing caspase activation and apoptosis or programmed necrosis.

In recent years, new monoclonal antibodies have been designed to target immune cytotoxicity to tumor cells. Examples include Rituxan (Rituximab, MabThera), which is designed for CD20-positive non-Hodgkin B cell lymphoma, and B-CLL and Herceptin (trastuzumab) for breast tumors overexpressing the human epidermal growth factor receptor 2 (HER-2). The clinical and commercial success of such anticancer antibodies has created great interest in antibody-based therapeutics for hematopoietic malignant neoplasms and solid tumors. However, due to the increased resistance of cancer cells to complement-mediated lysis, the clinical impact of these antibodies has been restricted to activation of antibody-mediated cell cytotoxicity (ADCC) responses and activation of programmed cell death (apoptosis). Augmentation of the complement-mediated cytotoxic (CDC) activity of these antibodies is likely to amplify the therapeutic potential of tumor-targeted antibodies.

The membrane complement regulatory proteins (mCRPs) in plasma and on cells [e.g., the decay accelerating factor (DAF, CD55), membrane cofactor protein (MCP, CD46) and CD59] control complement activation and prevent unnecessary complement consumption and attack of innocent cells (e.g., prevent cell lysis). Tumor cells are protected from complement due to over-expression of the mCRPs. For example, lung cancer cells over-express CD55 and CD46 and are consequently complement resistant relative to normal primary lung tissue. Similarly, the level of CD59 expression in colorectal carcinoma correlates with the degree of differentiation and progression of the disease. Neutralization of the membrane regulators by specific monoclonal antibodies increases complement-mediated lysis of tumor cells (Fishelson Z. et al. Mol. Immunol. 40: 109-123, 2003; Donin N. et al. Clin. Exp. Immunol. 131: 254-263, 2003).

Other protective mechanisms against complement attack include activation of certain protein kinase cascades, synthesis of new proteins, and activity of heat shock proteins and Bcl-2. In addition, cells exposed to sublytic doses of complement become, within 60 minutes, increasingly resistant to lytic complement doses. One of the outcomes of these cell activation processes is a rapid removal of the MAC from the cell surface by vesiculation (“budding-out”) or endocytosis and proteolytic fragmentation. Physical removal of the MAC by vesiculation is a general phenomenon demonstrated in a large number of cell types including neutrophils, oligodendrocytes, glomerular epithelial cells, U937 and K562 cells. During the vesiculation process, sorting of membrane proteins and lipids occurs and the shed vesicles are enriched with cholesterol, diacylglycerol and the C9 protein of the MAC complex. The protection against complement-mediated lysis by the removal of MAC from the cell surface requires calcium ions and active protein phosphorylation reactions (Kraus S and Fishelson Z, 2000, Eur. J. Immunol. 30:1272-1280).

Mortalin (also known as GRP75, PBP74 and mitochondrial hsp70) is found in the cytoplasm, ER and cytoplasmic vesicles, yet its prime location is in mitochondria. Several functions have been attributed to mortalin including stress response, glucose regulation, intracellular trafficking, p53 inactivation, control of cell proliferation, tumorigenesis and import of proteins into mitochondria. Thus, mortalin is frequently upregulated in tumors, its over expression in normal cells considerably extends their lifespan, while reduced mortalin levels in immortalized cells causes growth arrest.

The present inventors have recently identified Mortalin as part of the MAC-induced vesicles released from K562 human erythroleukemic cells and demonstrated that it is shed from the cells after activation of the complement system (Pilzer D and Fishelson Z, 2005, Int. Immunol. 17: 1239-1248). In addition, U.S. patent application Ser. No. 11/440,132 to the present inventors discloses methods of decreasing the level of mortalin (e.g., using an anti-mortalin antibody or siRNA) for decreasing vesicular shedding of complement and increasing complement-mediated cytolysis of pathological cells in diseases such as cancer and pathogenic infection.

MKT-077, a cationic rhodacyanine dye analogue, binds to mortalin and abrogates its interactions with p53 thus rescues its transcriptional activation function [Wadhwa, R., et al., Cancer Res., 2000. 60: 6818-21]. Due to its positive charge, MKT-077 can pass through membrane lipid bilayers and accumulate inside mitochondria, which carry a high negative charge. Cancer cells, which have a higher mitochondrial membrane potential than normal cells, show higher sensitivity to MKT-077. This selective toxicity for cancer cells has led to the use of MKT-077 in preclinical and clinical cancer therapeutic trials. However, MKT-077 was found to be toxic (renal toxicity) when administered as a five-day infusion (30-50 mg/m²/day) once every three weeks in a phase I clinical trial of chemo-resistant solid tumors (Propper D. J. et al., 1999. Ann. Oncol., 10: 923-927). Toxicity (as a manageable hypomagnesemia) was also encountered when MKT-077 was administered as a single bolus weekly infusion (42 mg/m²) for 4 weeks every 6 weeks (Britten C D., et al., 2000, Clin. Cancer Res. 6: 42-49). Other adverse effects of MKT-077 include reversible impairment of mitochondrial function, cross-linking of actin filaments and inactivation of telomerase. For all these reasons the use MKT-077 as an anti-cancer drug was deemed undesirable and development of MKT-077 was discontinued.

### SUMMARY OF THE INVENTION

According to one aspect of the present invention there is provided a method of treating cancer in a subject in need thereof, the subject being treated with an anti-cancer antibody therapy, the method comprising administering to the subject a therapeutically effective amount of a delocalized lipophilic cation (DLC) compound capable of binding mortalin, thereby treating the cancer in the subject.

According to another aspect of the present invention there is provided use of a delocalized lipophilic cation (DLC) compound capable of binding mortalin for the manufacture of a medicament identified as an adjuvant for an anti-cancer antibody therapy.

According to yet another aspect of the present invention there is provided an article of manufacture comprising a packaging material which comprises a label for use as an adjuvant anti-cancer antibody therapy, the packaging material packaging a delocalized lipophilic cation (DLC) compound capable of binding mortalin.

According to still another aspect of the present invention there is provided a pharmaceutical composition comprising as an active ingredient a delocalized lipophilic cation (DLC) compound and an anti-cancer antibody, the delocalized lipophilic cation (DLC) being capable of binding mortalin, and a pharmaceutically acceptable carrier.

According to an additional aspect of the present invention there is provided a method of identifying a delocalized lipophilic cation (DLC) adjuvant for anti-cancer antibody therapy, comprising: (a) contacting cells expressing mortalin with a plurality of delocalized lipophilic cation (DLC) compounds, and; (b) identifying at least one compound from the plurality of compounds which is capable of down-regulating a mortalin function, the at least one compound being the adjuvant for anti-cancer antibody therapy.

According to yet an additional aspect of the present invention there is provided a unit dosage form comprising 0.17-220 milligram of MKT-077.

According to further features in the embodiments of the invention described below, the packaging material further packaging an anti-cancer antibody therapy.

According to still further features in the described embodiments, administering the delocalized lipophilic cation (DLC) is effected prior to administration of the anti-cancer antibody therapy.

According to still further features in the described embodiments, administering the delocalized lipophilic cation (DLC) is effected concomitant with the administration of the anti-cancer antibody therapy.

According to still further features in the described embodiments, the delocalized lipophilic cation (DLC) and the anti-cancer antibody are packaged in separate containers.

According to still further features in the described embodiments, the delocalized lipophilic cation (DLC) and the anti-cancer antibody are packaged in a single container.

According to still further features in the described embodiments, the delocalized lipophilic cation (DLC) and the anti-cancer antibody are co-formulated.

According to still further features in the described embodiments, the delocalized lipophilic cation (DLC) is a cationic rhodacyanine compound.

According to still further features in the described embodiments, the cationic rhodacyanine compound is MKT-077.

According to still further features in the described embodiments, the anti-cancer antibody is selected from the group consisting of Rituximab, Trastuzumab, Gemtuzumab, ozogamicin, Alemtuzumab, Ibritumomab tiuxetan, Tositumomab, Cetuximab, Bevacizumab, CP-751,871 and Panitumumab.

According to still further features in the described embodiments, the anti-cancer antibody therapy is directed against a tumor associated antigen selected from the group consisting of CD20, HER2, CD33, CD52, EGFR and IGF1R.

According to still further features in the described embodiments, the MKT-077 is formulated for intravenous administration.

According to still further features in the described embodiments, the administration of the MKT-077 is effected at a dosage of 0.1-25 mg/m²/day.

According to still further features in the described embodiments, the administration of the MKT-077 is effected at a dosage of 1-126 mg/m²/week.

According to still further features in the described embodiments, the anti-cancer antibody therapy is RITUXAN and whereas administration of the anti-cancer antibody is effected at a dosage of 375 mg/m².

According to still further features in the described embodiments, the mortalin function comprising binding to a complement protein.

According to still further features in the described embodiments, the complement protein is a C9 protein as set forth by SEQ ID NO:9.

Embodiments of the invention provide methods and pharmaceutical compositions for treating cancer.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. In case of conflict, the patent specification, including definitions, will control. In addition, the materials, methods, and examples are illustrative only and not intended to be limiting.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of biotechnology.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a histogram depicting the sensitization of cells to complement mediated lysis by MKT-077. K562 cells were pretreated for 1 hour at 37° C. with increasing concentrations of MKT-077 [or double distilled water (DDW); NT] and then treated with a sublytic dose of anti-K562 antibody (1:14) and complement [Normal human serum (NHS)] or heat-inactivated NHS (HI-NHS). After incubation for 1 hour at 37° C., percent cell lysis was measured with 0.2% Trypan Blue. Results present the percentage of cell lysis and are representative of four independent experiments. P<0.01 between NT group and the other groups.

FIGS. 2a-b are Western Blot analyses demonstrating that MKT-077 prevents release of mortalin and C9 from complement attacked cells. K562 cells pretreated with 50 μM MKT-077 (or with double distilled water) were treated for 10 minutes at 37° C. with a sublytic dose of anti-K562 antibody and NHS or HI-NHS. Then, the cells were washed 4 times with Hank's Balanced Salt Solution (HBSS; Sigma, Rehovot, Israel), incubated for 10 minutes at 37° C. and supernatant were collected. Released proteins were first sedimented at 5,000 g for 15 minutes and then centrifuged at 100,000 g for 30 minutes for vesicles isolation. High-speed supernatant and pellets were analyzed by SDS-PAGE and Western Blotting with anti-mortalin (FIG. 2a) or anti-C9 (FIG. 2b) antibodies. Results are representative of three independent experiments.

FIG. 3 is a histogram depicting that the effect of MKT-077 on complement-mediated lysis is p53-independent. HCT116 colon carcinoma cell line (HCT) p53 +/+ and HCT p53 −/− cells were pretreated for 1 hour at 37° C. with 50 μM MKT-077 and then treated with a lytic dose of anti-K562 antibody (1:9) and NHS or HI-NHS. After incubation for 1 hour at 37° C., percent cell lysis was measured by Trypan blue inclusion. Results present the percentage of cell lysis and are representative of three independent experiments. Differences between groups with and without MKT-077 or with and without p53 were statistically significant. *, P<0.01.

FIGS. 4a-b are histograms demonstrating that MKT-077 has no effect on SLO-mediated lysis and A23187-mediated lysis. FIG. 4a—K562 cells were pretreated for 1 hour at 37° C. with 50 μM MKT-077 and then treated with 900 and 1200 units ml⁻¹ SLO or with DTT as control. After 15 minutes at 37° C., cell lysis was measured by Trypan blue inclusion. Results are representative of three independent experiments. FIG. 4b—K562 cells were treated for 1 hour at 37° C. with increased concentrations of A23187 and with 50 μM MKT-077. Cell lysis was then measured by Trypan blue inclusion. Results present the percentage of cell lysis and are representative of three independent experiments.

FIG. 5 is a histogram depicting the interference of MKT-077 with binding of mortalin to C9. Microtiter plate wells were coated overnight with C9 or BSA (0.5 μg per well) and then incubated with K562 cell lysate (40×10⁶ cells ml⁻¹ lysis buffer) diluted 1:25 with or without 50 μM MKT-077. The wells were washed and treated with anti-mortalin antibody and peroxidase-labeled second antibody, developed with o-phenilenediamine (OPD) and analyzed in an ELISA reader. Results present the quantity of mortalin in each experimental group and are representative of three independent experiments. *,P<0.005 between groups with and without MKT-077.

FIG. 6 is a histogram depicting cell lysis experiments demonstrating that MKT-077, but not Rhodamine123, sensitize cells to complement. K562 cells were treated for 60 minute at 37° C. with different concentrations of MKT-077 or Rhodamine123 (Rho123), followed by treatment for another 60 minutes with anti-K562 antibodies and NHS or HI-NHS and the cell lysis was measured by Trypan blue inclusion. Results are representative of three independent experiments. *P<0.05, between cells treated with NHS and MKT-077 and cells treated with NHS without MKT-077.

FIGS. 7a-e schematically depict the general formulas I-V of the DLC compounds of the invention (e.g., rhodacyanine compounds).

FIG. 7a—The rhodacyanine compound is represented by general Formula I, the cation moiety of which has a log P value of 4.5-12: wherein X₁ and X₂, which may be the same or different, each represents O, S, Se, —CH═CH—, formula VI

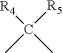

or formula VII

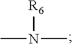

Y₁ represents O, S, Se, or formula VIII

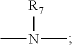

R₁ and R₃, which may be the same or different, each represents an alkyl group; R₂ represents an alkyl group, an aryl group or a heterocyclic group; Z₁ and Z₂, which may be the same or different, each represents an atomic group necessary to form a 5- or 6-membered ring; L₁, L₂ and L₃, which may be the same or different, each represents a methine group or nitrogen atom and L₁ and R₃ may combine and form a 5- or 6-membered ring; R₄ and R₅, which may be the same or different, each represents an alkyl group; R₆ and R₇, which may be the same or different, each represents an alkyl group or an aryl group; Q represents a pharmaceutically acceptable anion; j and k, which may be the same or different, each represents 1 or 2; m and n, which may be the same or different, each represents 0 or 1;

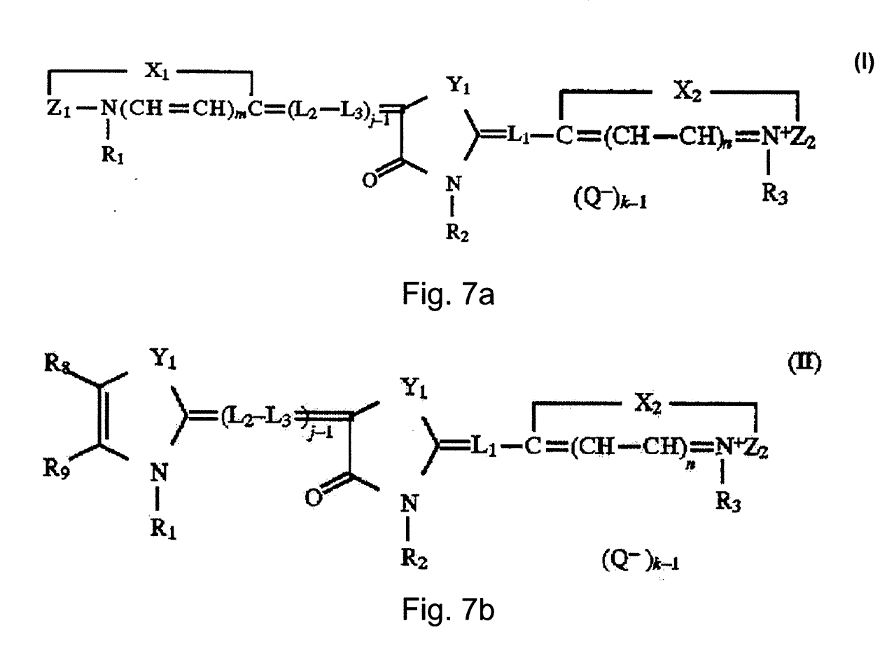

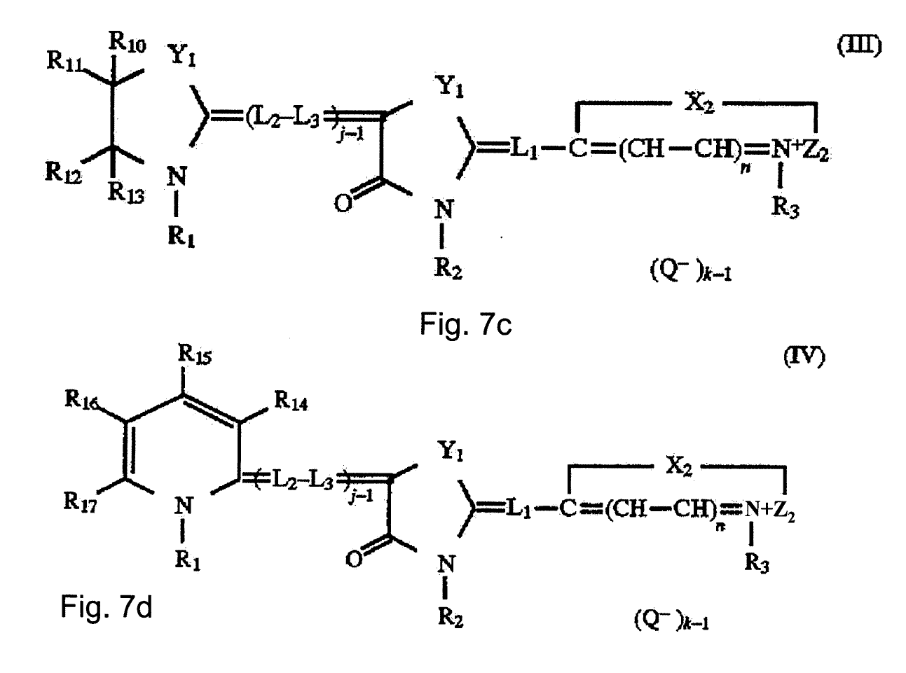

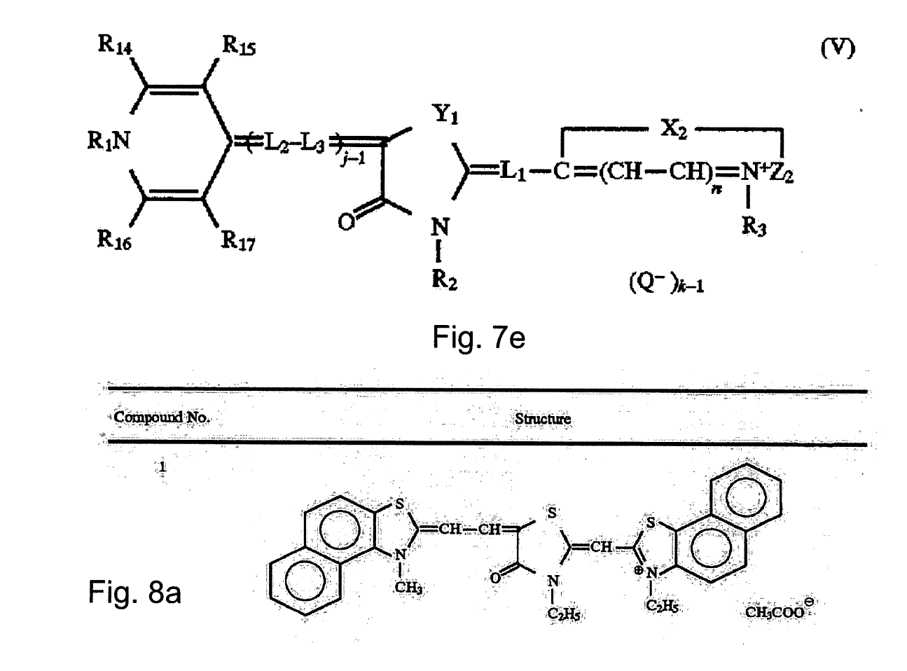

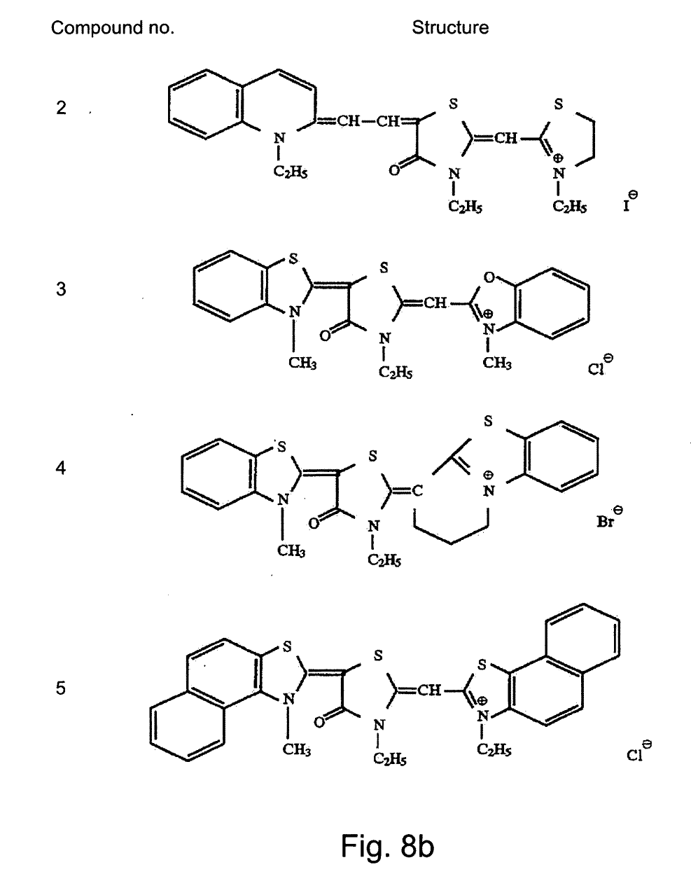

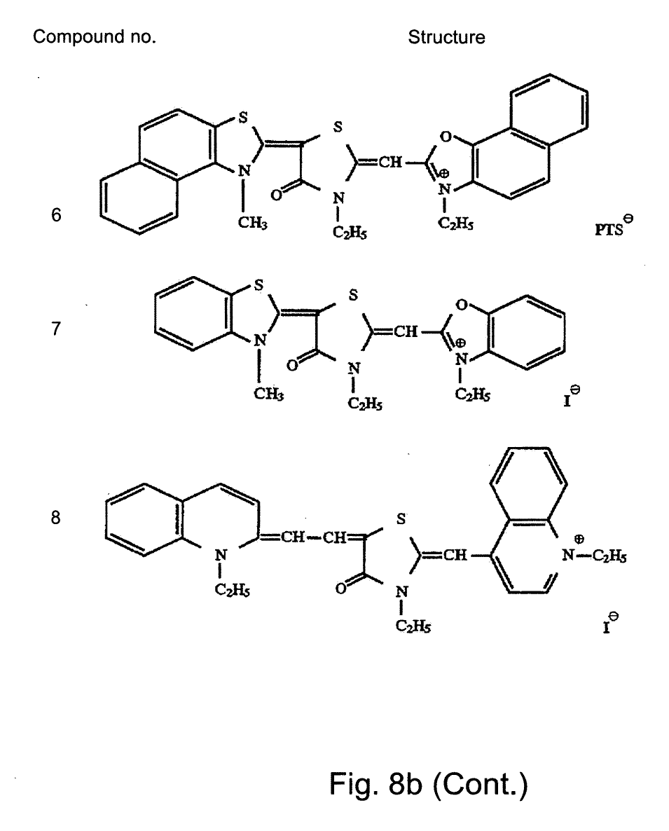

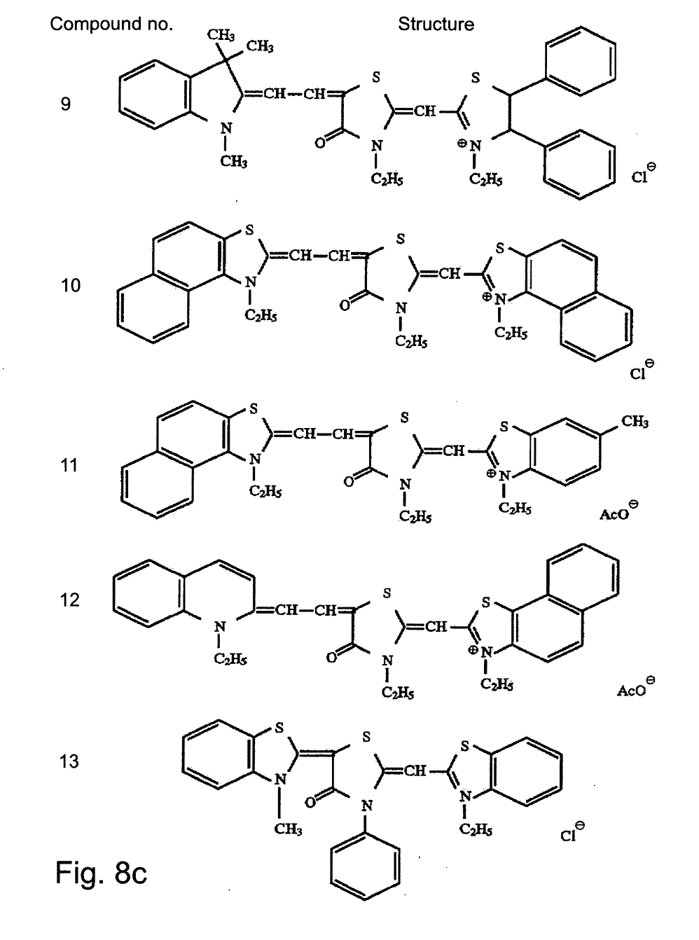

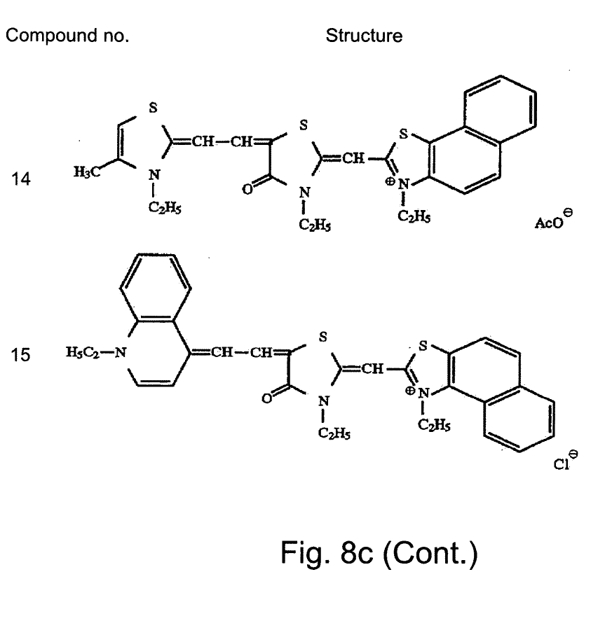

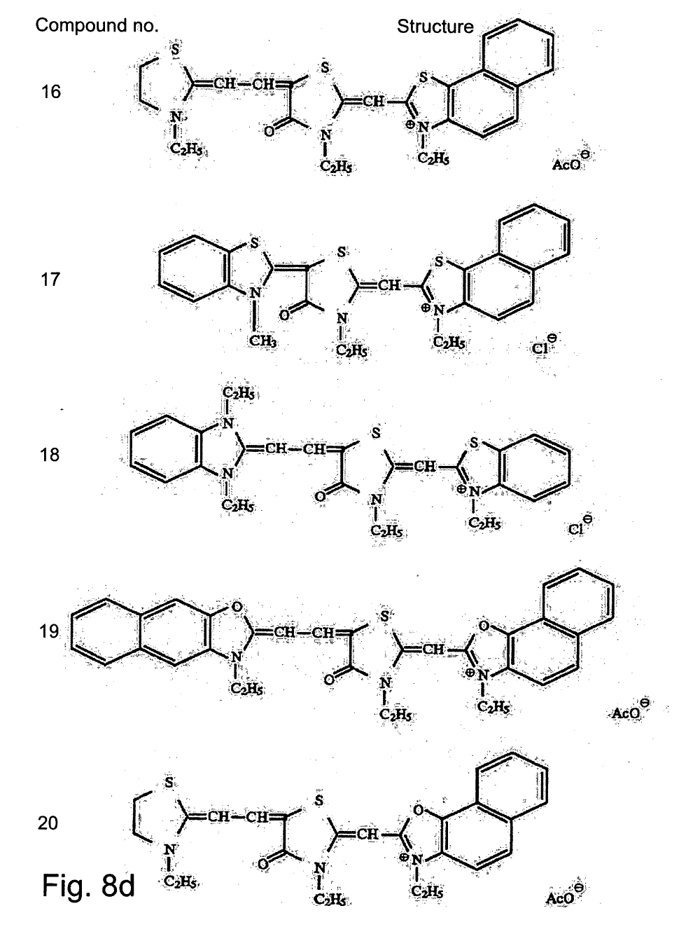

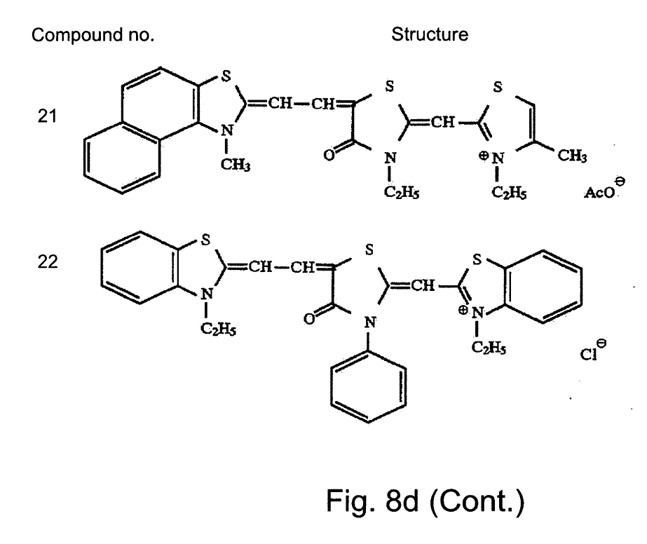

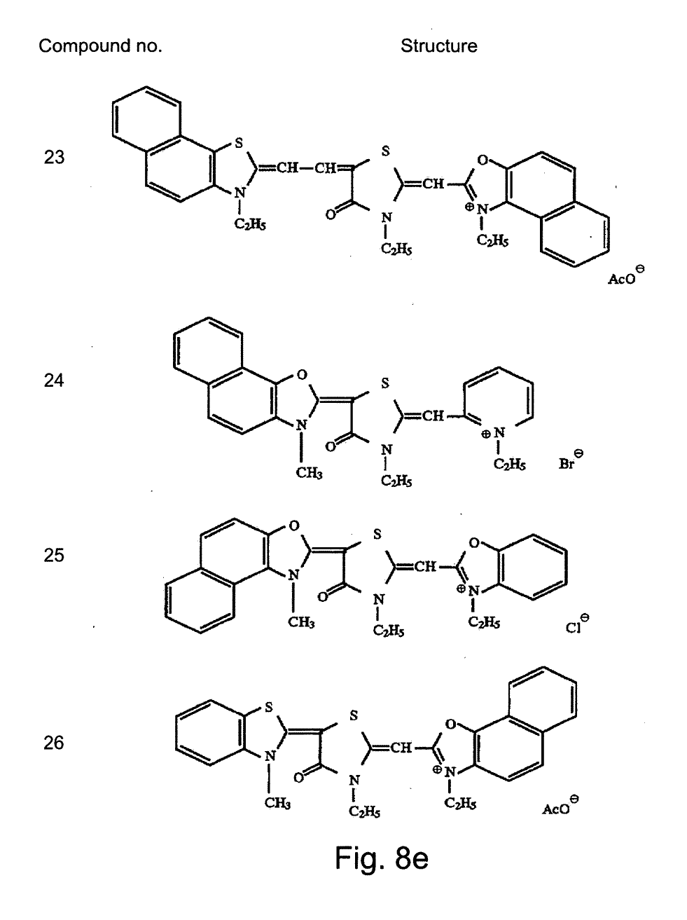

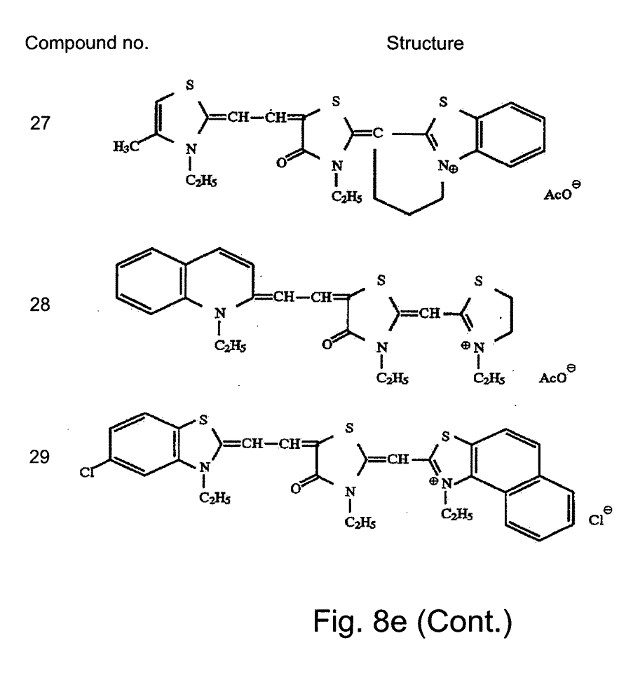

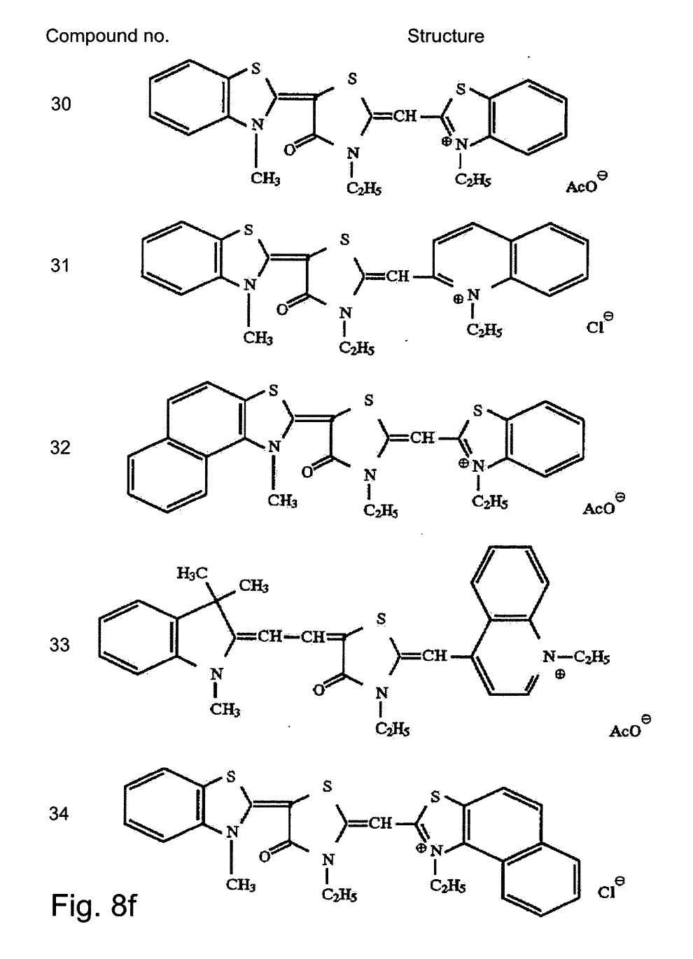

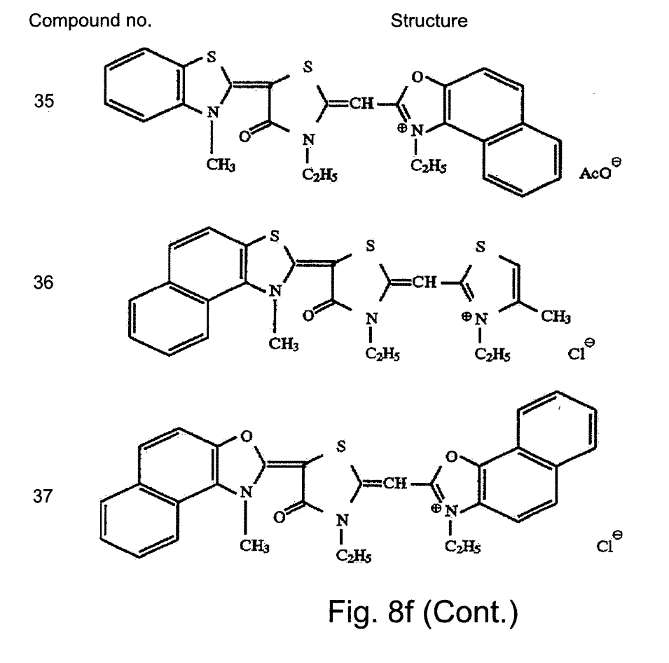

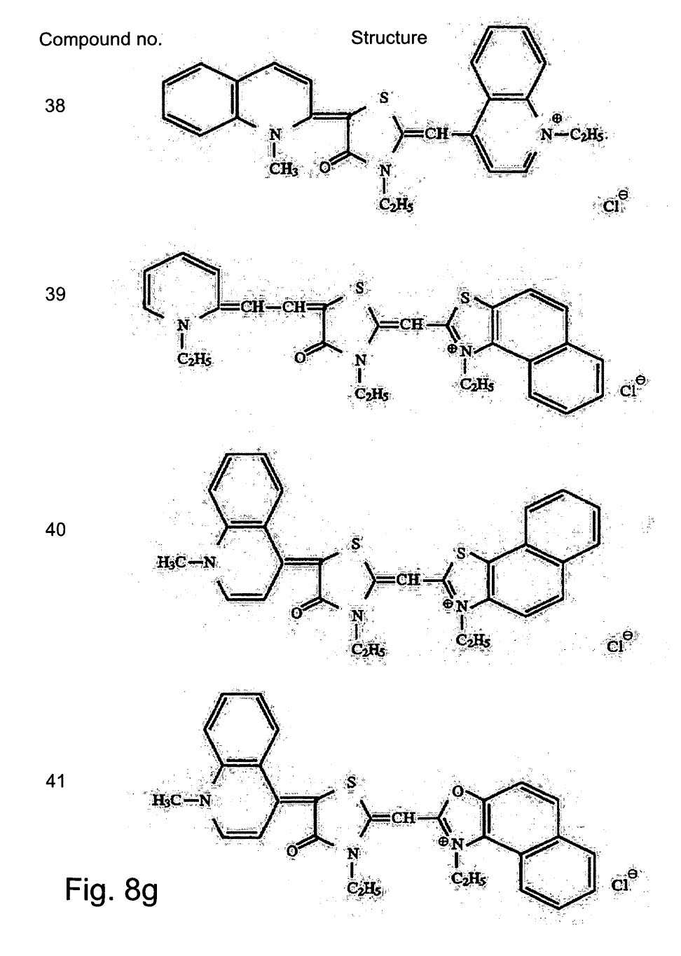

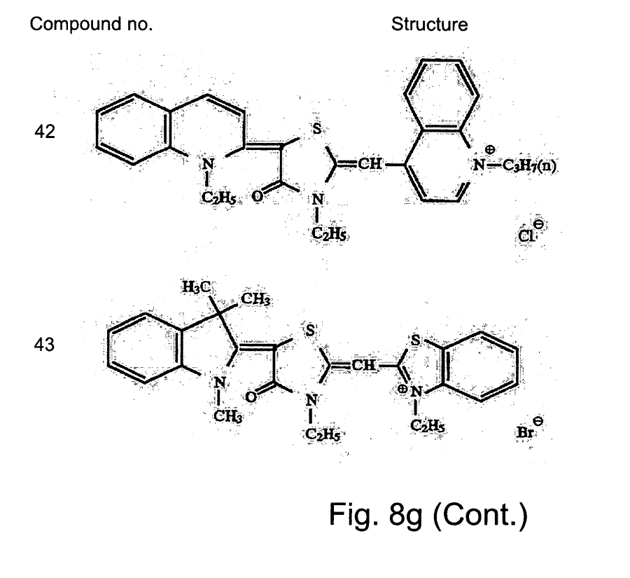

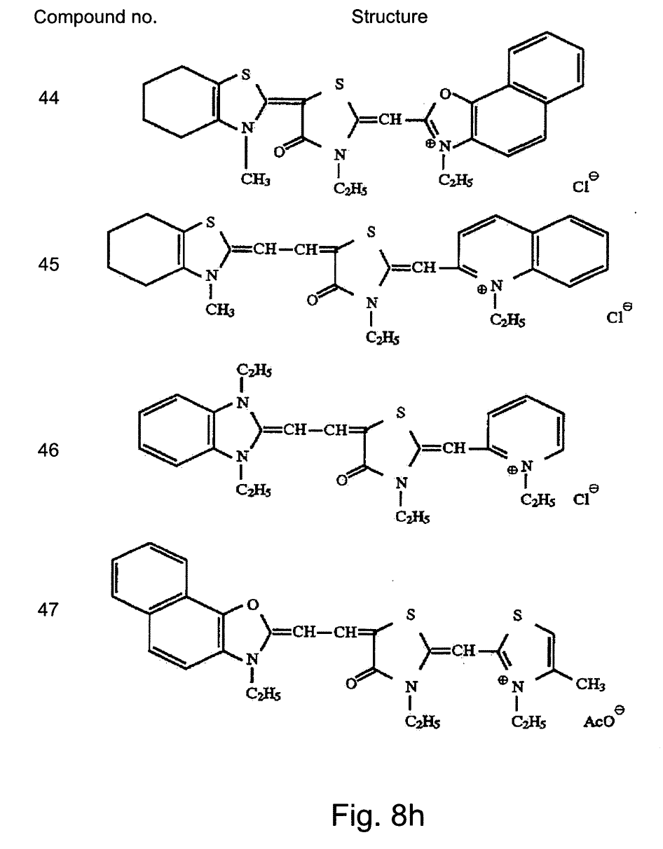

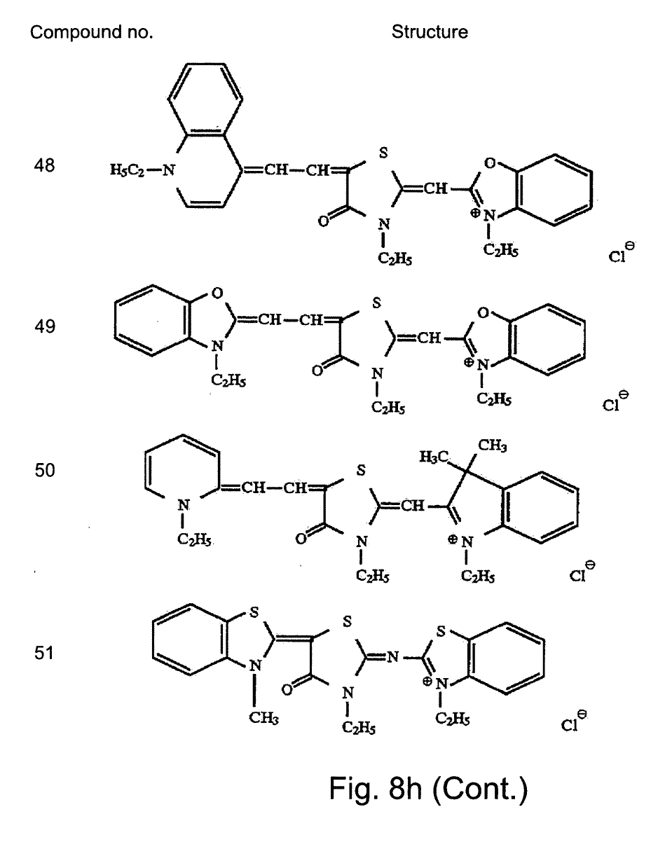

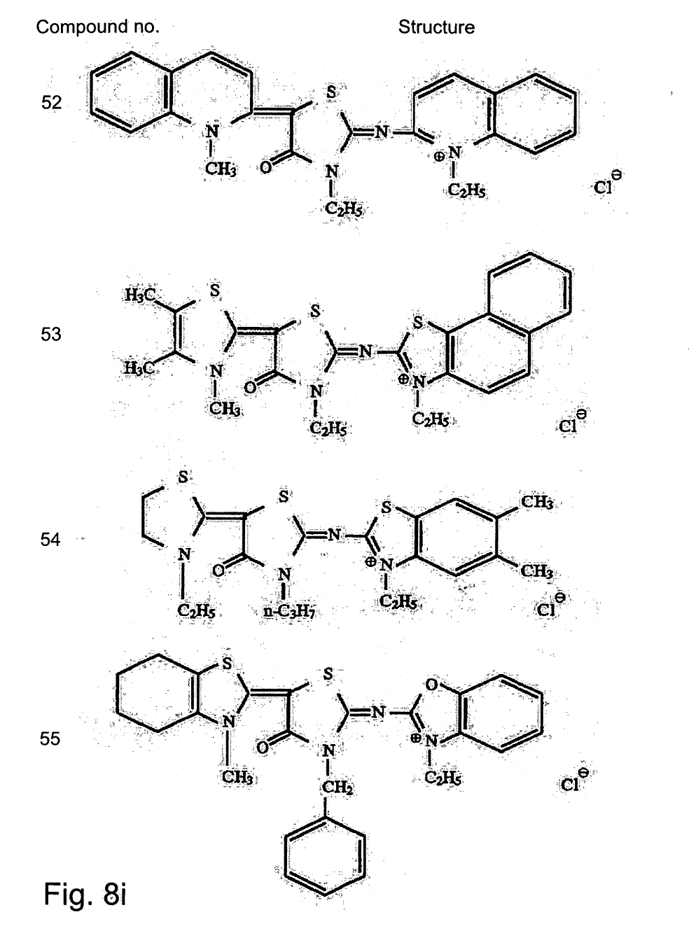

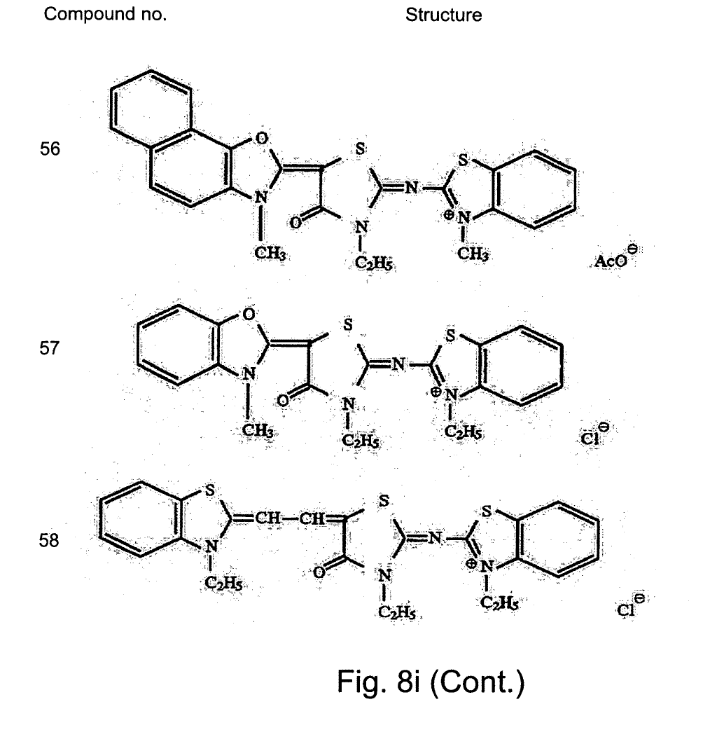

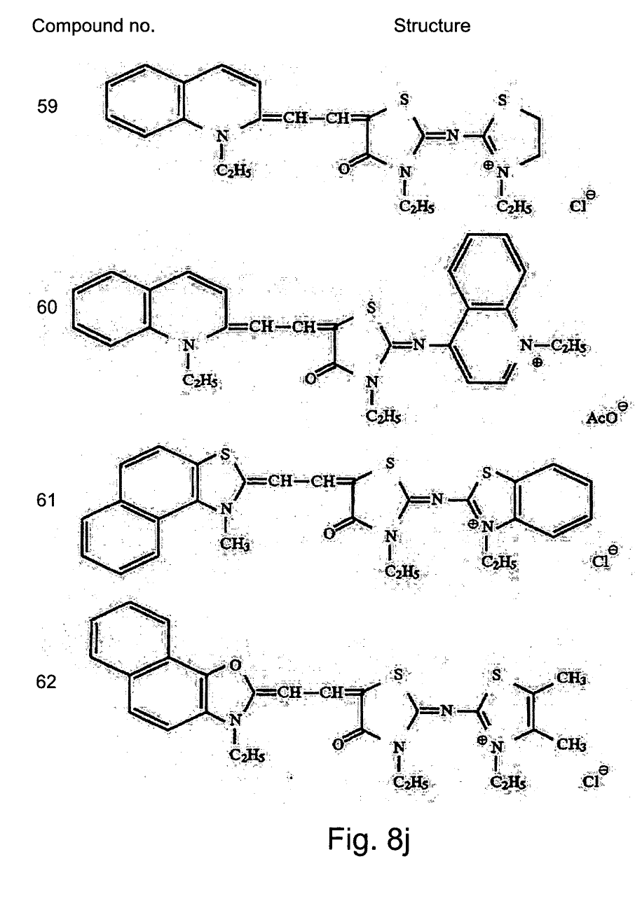

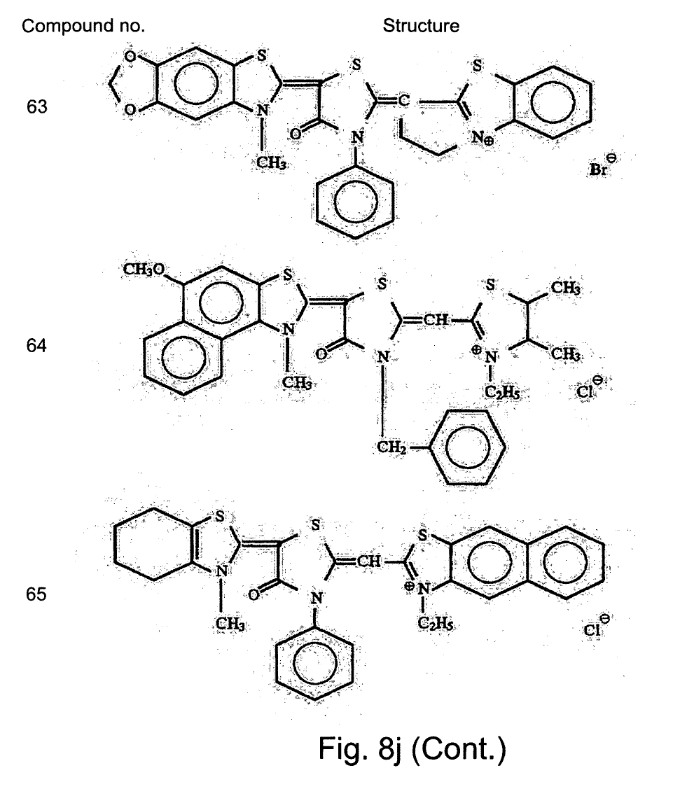

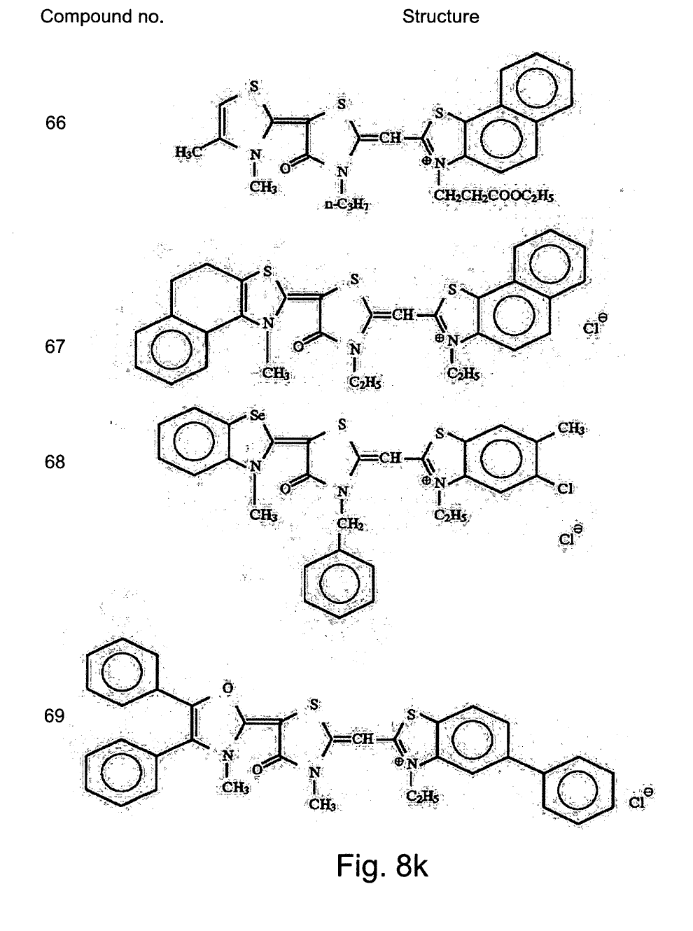

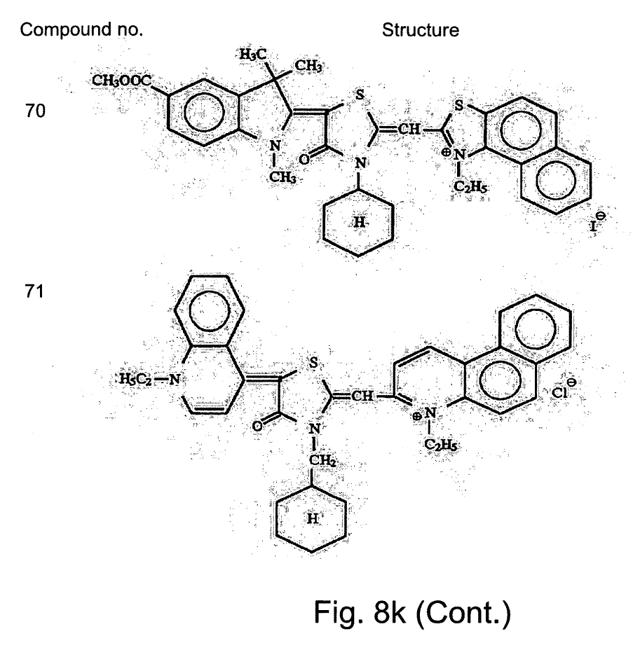

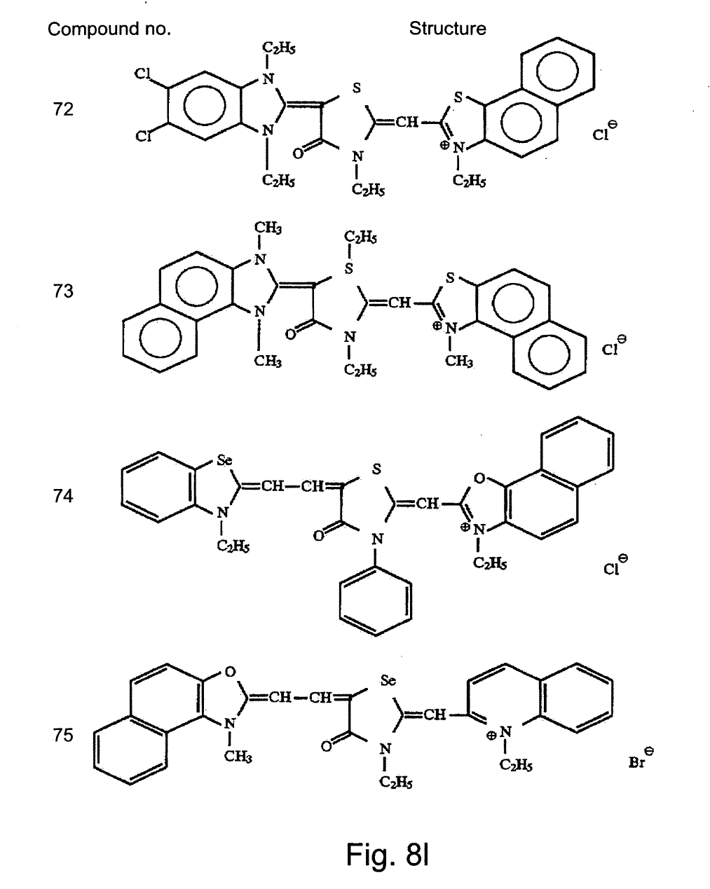

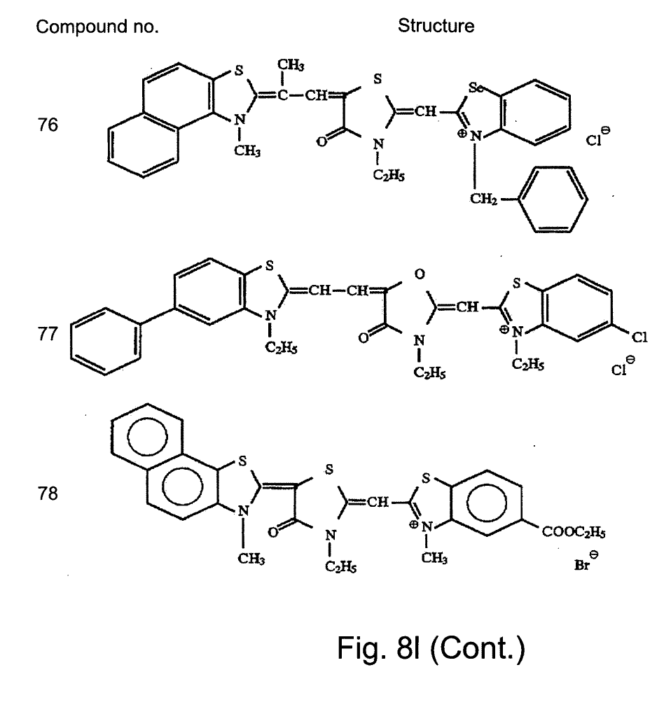

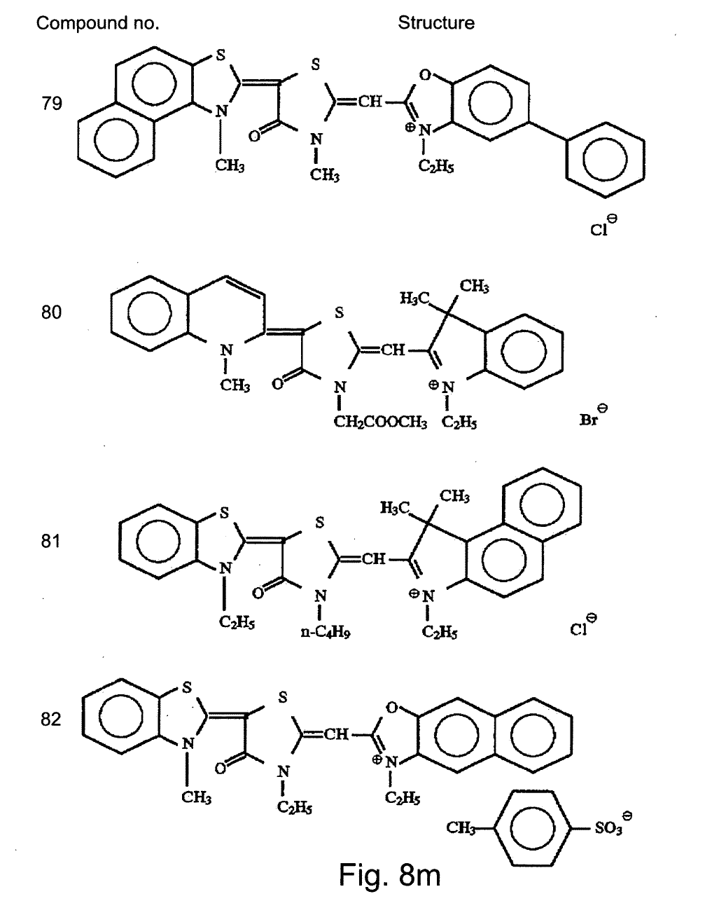

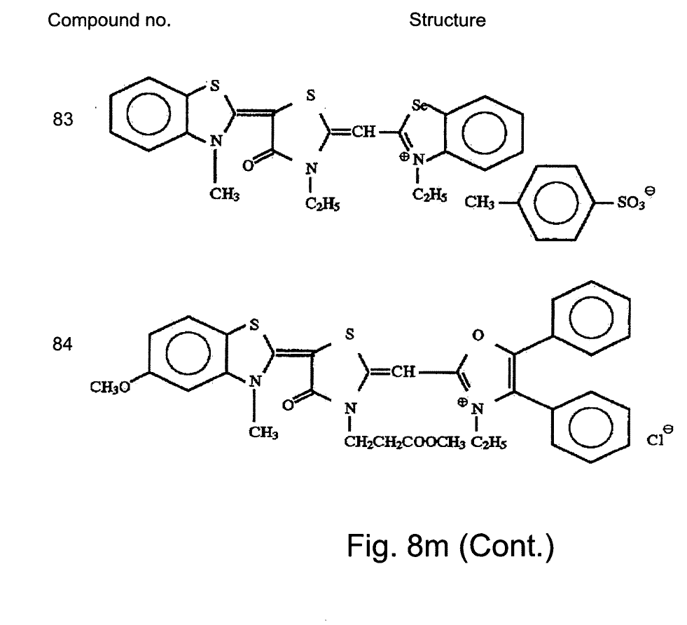

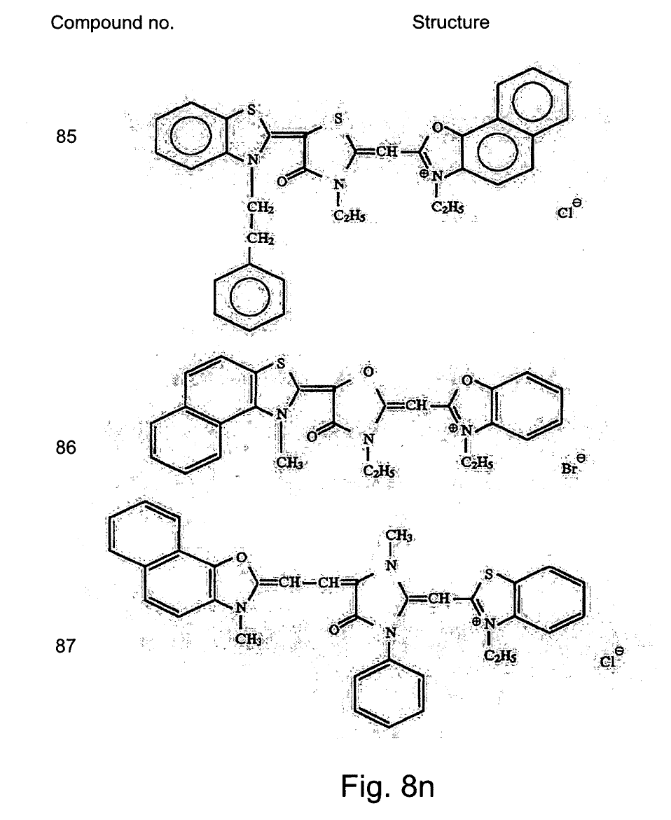

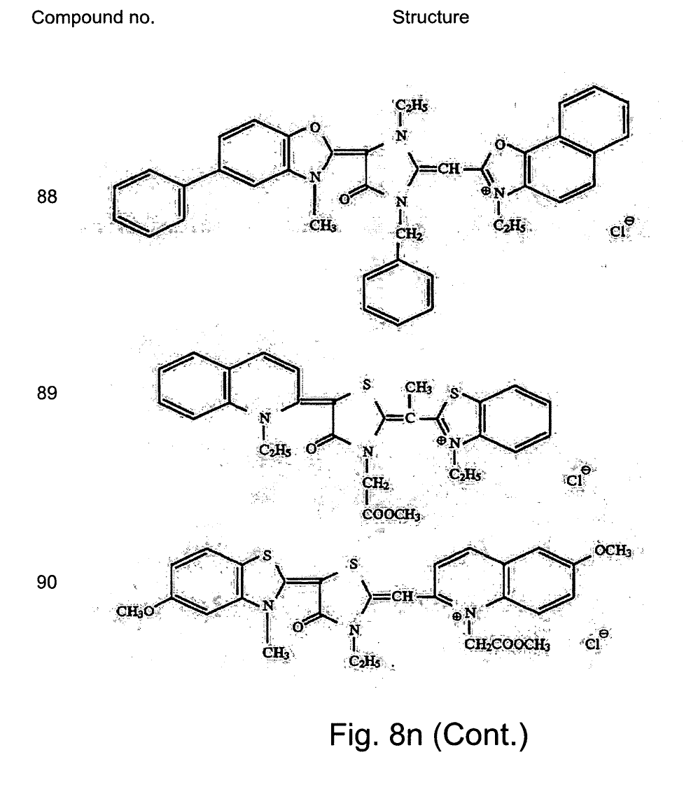

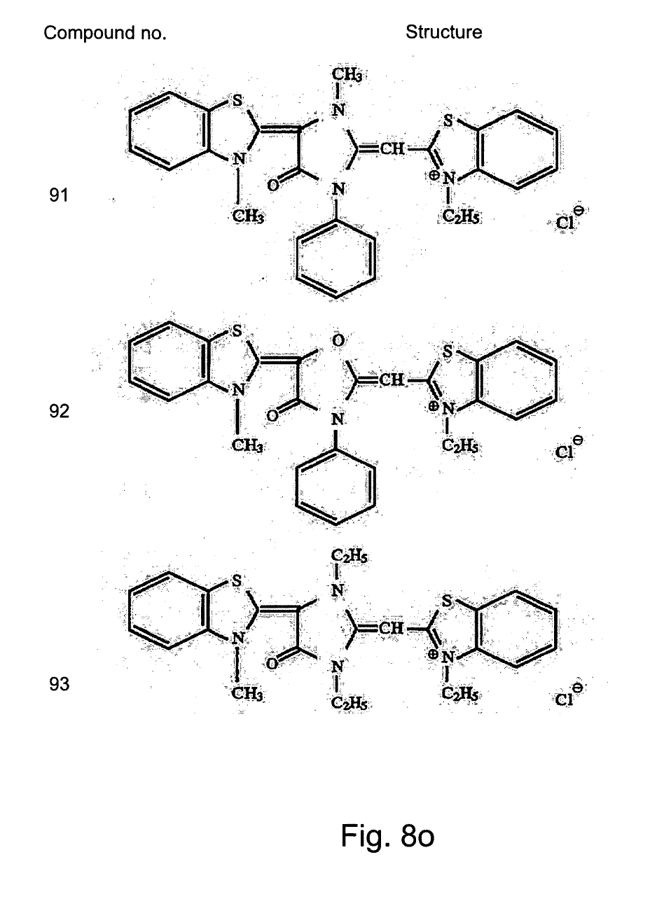

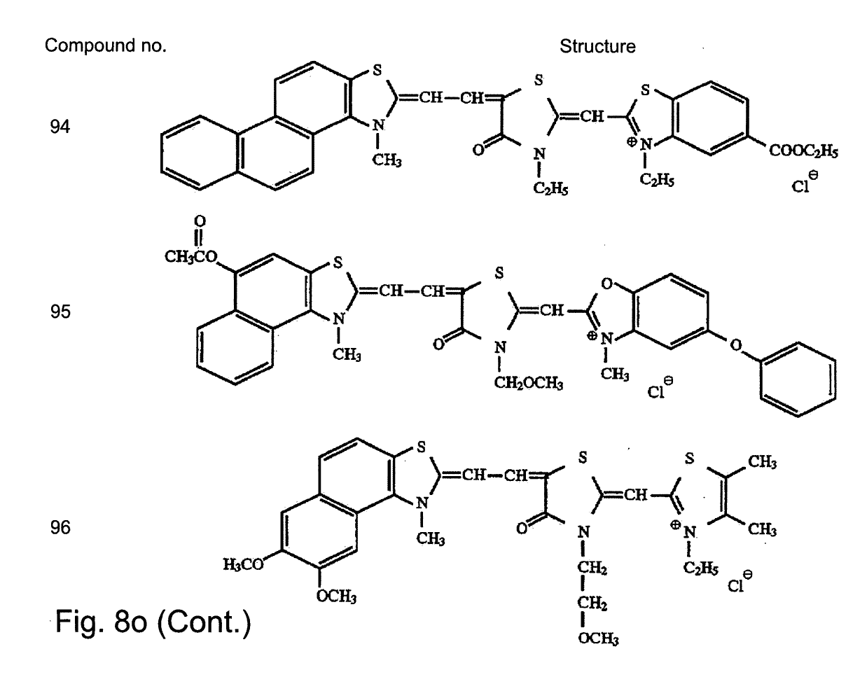

FIG. 7b depicts rhodacyanine compounds of general Formulae (II), wherein Z₂, Y₁, X₁, X₂, R₁, R₂, R₃, L₁, L₂, L₃, Q, j, k and n all have the same meanings as defined above; R₈ and R₉, which may be the same or different, each represents a hydrogen atom, an alkyl group or an aryl group, or R₈ and R₉ may combine and form a fused 5- or 6-membered ring;

FIG. 7c depicts rhodacyanine compounds of general Formulae (III), wherein X₁, Y₁, X₂, Z₂, R₁, R₂, R₃, Q, j, k, L₁, L₂, L₃ and n have the same meanings as defined above; R₁₀, R₁₁, R₁₂ and R₁₃, which may be the same or different, each represents a hydrogen atom, an alkyl group or an aryl group, or any two of R₁₀ to R₁₃ may combine and form a 5- or 6-membered ring;

FIGS. 7d and 7e depict rhodacyanine compounds of general Formulas IV and V, respectively, wherein Y₁, X₂, Z₂, R₁, R₂, R₃, Q, j, k, L₁, L₂, L₃ and n have the same meanings as define above; R₁₄, R₁₅, R₁₆, and R₁₇, which may be the same or different, each represents a hydrogen atom, an alkyl group, an aryl group, an alkoxy group, an aryloxy group, an acyl group, an alkoxycarbonyl group, a benzoyl group, an ureido group, an amino group, an amido group, an sulfamido group, a carbomoyl group, a sulfamoyl group, a halogen atom, a nitro group, a cyano group, a hydroxy group or a carboxyl group, or any adjacent two of R₁₄ to R₁₇ may combine and form a 5- or 6-membered ring (for further description see U.S. Pat. No. 5,670,530).

FIGS. 8a-o depict exemplary DLC compounds of the invention. FIG. 8a—DLC compound 1; FIG. 8b—DLC compounds 2-8; FIG. 8c—DLC compounds 9-15; FIG. 8d—DLC compounds 16-22; FIG. 8e—DLC compounds 23-29; FIG. 8f—DLC compounds 30-37; FIG. 8g—DLC compounds 38-43; FIG. 8h—DLC compounds 44-51; FIG. 8i—DLC compounds 52-58; FIG. 8j—DLC compounds 59-65; FIG. 8k—DLC compounds 66-71; FIG. 8l—DLC compounds 72-78; FIG. 8m—DLC compounds 79-84; FIG. 8n—DLC compounds 85-90; FIG. 8o—DLC compounds 91-96;

### DESCRIPTION OF EMBODIMENTS OF THE INVENTION

The invention is of, in some embodiments, methods and pharmaceutical compositions comprising a delocalized lipophilic cation (DLC) compound as an adjuvant for anti-cancer antibody therapy. Specifically, the DLC compound of the invention is capable of sensitizing cancerous cells to complement-mediated cell lysis and thereby increasing the efficiency of anti-cancer antibody therapy.

The principles and operation of the methods, article-of-manufacturing and pharmaceutical compositions for treating cancer according to the invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

While reducing the invention to practice, the present inventors have uncovered that a delocalized lipophilic cation (DLC) compound (e.g., MKT-077) which is capable of binding mortalin can sensitize cancerous cells to complement-mediated cell lysis and thus can be used as an adjuvant in anti-cancer antibody therapy.

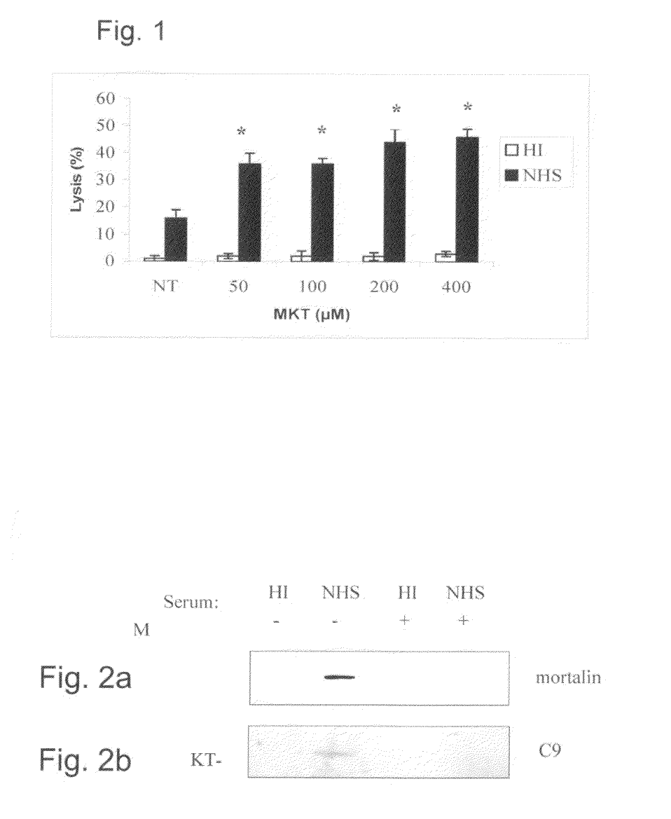

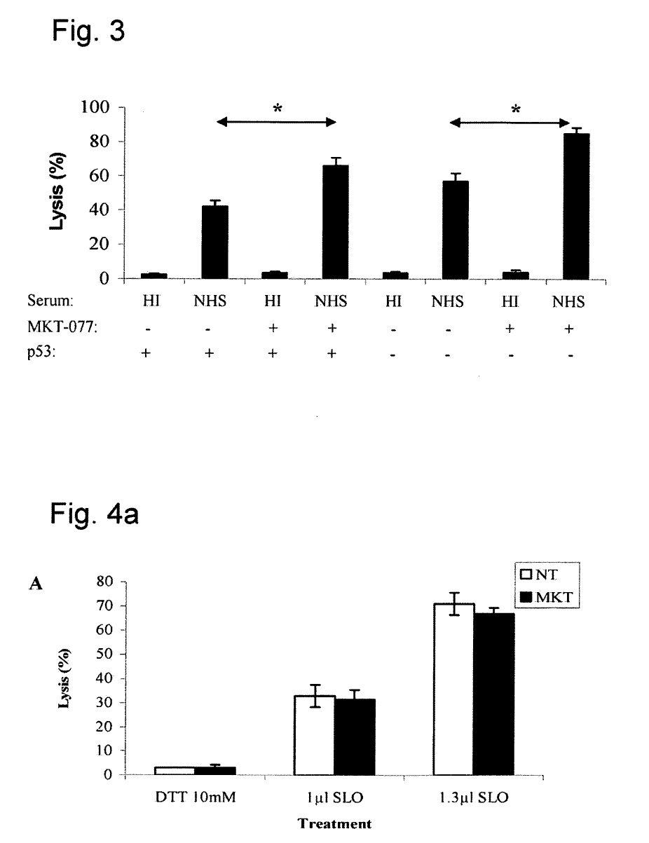

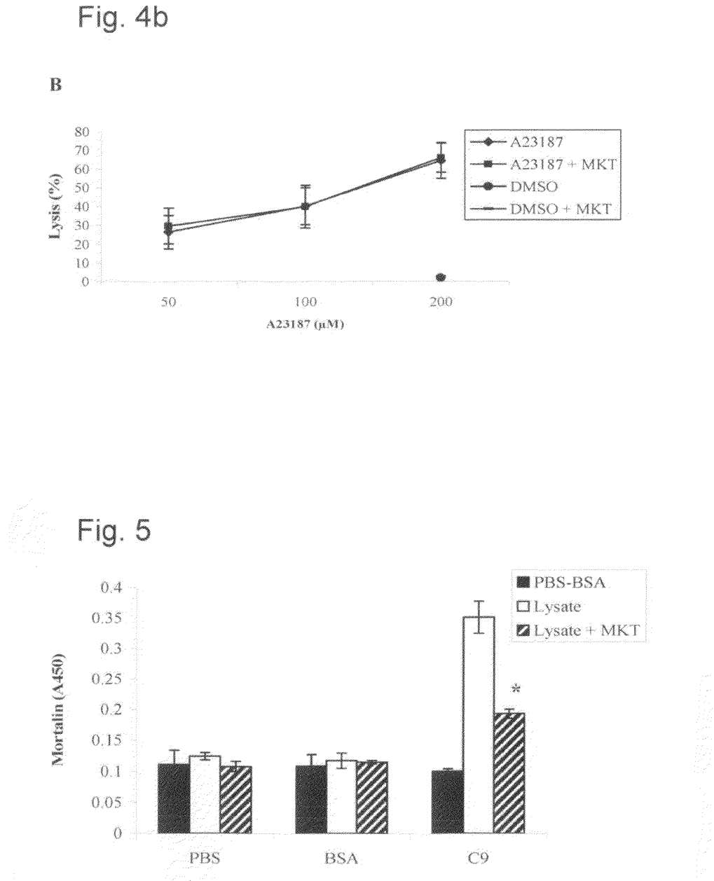

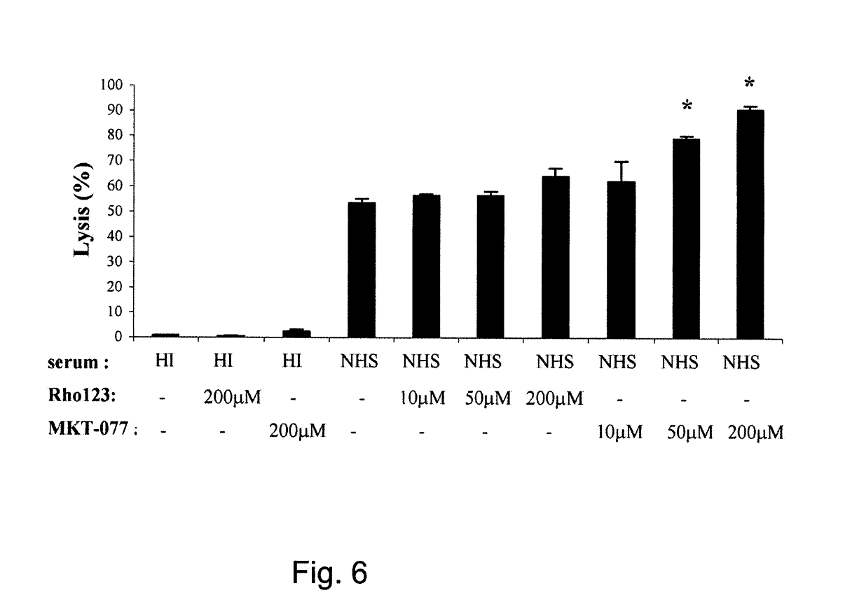

As shown in FIGS. 1-6 and described in Example 1 of the Examples section which follows, pre-treatment of human K562 erythroleukemia cells with MKT-077 prior to treatment with anti-K562 antibodies and complement (NHS) resulted in a significant enhancement of complement-mediated cell killing (FIG. 1), but not cell lysis induced by another pore-former, streptolysin O (FIG. 4a). MKT-077 sensitization was associated with inhibition of vesiculation of mortalin and C9 (FIGS. 2a-b) as well as with inhibition of the binding of mortalin to C9 of the MAC complex (FIG. 5). As is further shown in Example 1 of the Examples section which follows, the effect of MKT-077 on complement-mediated cell lysis is p53-independent (FIG. 3) and mortalin-mediated (FIGS. 4a-b). In addition, as is shown in FIG. 6, the sensitization effect of DLC compounds on complement-mediated cells lysis is specific to compounds capable of inhibiting mortalin such as MKT-077. These results demonstrate that MKT-077 interferes with the binding of mortalin to the MAC and, consequently, lowers the capacity of the cells to eliminate the MAC from their surface and to resist complement-mediated lysis. Thus, MKT-077 sensitizes cells to complement mediated cell lysis in a mortalin-dependent manner. Altogether, these results suggest the use of delocalized lipophilic cation (DLC) compounds such as MKT-077, which are capable of binding and optionally inhibiting mortalin as an adjuvant for anti-cancer antibody therapy.

Thus, according to one aspect of the invention there is provided a method of treating cancer in a subject in need thereof, the subject being treated with anti-cancer antibody therapy. The method is effected by administering to the subject a therapeutically effective amount of a delocalized lipophilic cation (DLC) compound capable of binding mortalin, thereby treating the cancer.

As used herein the phrase “adjuvant therapy” or “pharmaceutical adjuvant” refers to drugs (i.e., DLCs) or drug protocols that have few or no pharmacological effects by themselves, but may increase the efficacy or potency of other drugs (i.e., anti cancer antibody therapy) when given at the same time.

The term “treating” refers to inhibiting, preventing (i.e., keeping a pathology from occurring in a subject who may be at risk for the pathology) or arresting the development of a pathology (e.g., tumor, cancer or pre-cancerous tumor) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, preferably human beings at any age which suffer from the pathology. The term encompasses individuals who were diagnosed with the cancer, optionally received cancer therapy (e.g., a conventional chemotherapy or anti-cancer antibody therapy) and are in a remission state of the disease. It will be appreciated that the latter individuals may develop resistance to the conventional anti-cancer antibody therapy by over-expression of the membrane complement regulatory proteins (mCRPs) and/or mortalin. Subjects of the invention are treated with anti-cancer antibody therapy or are candidates for such a treatment.

As used herein the term “mortalin” refers to the nucleic acids and/or amino acids of a mammalian mortalin such as the human mortalin [GenBank Accession No. NP_{—}004125.3 (SEQ ID NO:1, for amino acids) and NM_{—}004134.4 (SEQ ID NO:2, for the nucleic acids)], mouse mortalin (GenBank Accession Nos. NM_{—}010481.1 and NP_{—}034611.1) rat mortalin (GenBank Accession No. XR_{—}005519.1) and dog mortalin (Gene ID:474697).

As used herein the phrase “delocalized lipophilic cation (DLC) compound” refers to any π electron-delocalized lipophilic cations which is capable of binding mortalin and optionally inhibiting its activity and/or interfering (inhibiting) with the binding of mortalin to members of the MAC complex as the C9 protein (e.g., SEQ ID NO:9).

Examples of delocalized lipophilic cation (DLC) compounds (e.g., rhodacyanine compounds) which can be used in accordance to this aspect of the invention are listed in U.S. Pat. No. 5,670,530, which is incorporated by reference in its entirety, and include rhodacyanine compounds of general formulas I-V as depicted in FIGS. 7a-e.

In greater detail, in the Formulae (I) to (V) which are depicted in FIGS. 7a-e X₁ and X₂, individually, represents an oxygen atom, a sulfur atom, a selenium atom, formula VI

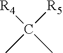

—CH═CH— or a group of formula VII

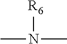

where R₄ and R₅ each represents an alkyl, i.e., an unsubstituted or substituted alkyl group such as a straight-chain, branched chain or cyclic alkyl group, and R₆ is an alkyl, i.e., an unsubstituted or substituted alkyl group such as a straight-chain, branched chain or cyclic alkyl group, an aryl, i.e. an unsubstituted or substituted aryl group such as a monocyclic or bicyclic aryl group, or a heterocyclic, i.e., an unsubstituted or substituted heterocyclic group such as a 5- to 6-membered heterocyclic group which tan be saturated or unsaturated and can contain one or more nitrogen atoms, oxygen atoms and sulfur atoms.

Y₁ represents an oxygen atom, a sulfur atom, a selenium atom or a group of formula VIII

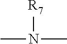

where R₇ is an alkyl, i.e., an unsubstituted or substituted alkyl group such as a straight-chain, branched chain or cyclic alkyl group, an aryl, i.e., an unsubstituted or substituted aryl group such as a monocyclic or bicyclic aryl group, or a heterocyclic group, i.e., an unsubstituted or substituted heterocyclic group such as a 5- to 6-membered heterocyclic group which can be saturated or unsaturated and can contain one or more nitrogen atoms, oxygen atoms and sulfur atoms.

R₁, R₂ and R₃ each individually represents an alkyl, i.e., an unsubstituted or substituted alkyl group such as a straight-chain, branched chain or cyclic alkyl group and R2 can additionally be an aryl, i.e., an unsubstituted or substituted aryl group such as a monocyclic, bicyclic or tricyclic aryl group or a heterocyclic, i.e., an unsubstituted or substituted heterocyclic group such as a 5- to 6-membered heterocyclic group which can be saturated or unsaturated and can contain one or more nitrogen atoms, oxygen atoms and sulfur atoms as hetero atoms.

Z₁ and Z₂, which may be the same or different, each represents an atomic group necessary to form a saturated or unsaturated 5- or 6-membered ring which may contain one or more nitrogen atoms, oxygen atoms, sulfur atoms or selenium atoms as hetero atoms and Z₁ and Z₂ may be each substituted or condensed with another ring such as a saturated or unsaturated ring.

L₁, L₂ and L₃ each represents a methine group, i.e., an unsubstituted or substituted methine group or nitrogen atom and when L₁ is a substituted methine group, L₁ and R₃ may combine to form a saturated or unsaturated 5- or 6-membered ring.

R₈ and R₉ each represents a hydrogen atom or an alkyl, i.e., an unsubstituted or substituted alkyl group such as a straight-chain, branched chain or cyclic alkyl group and moreover, R₈ and R₉ represents an aryl, i.e., an unsubstituted or substituted aryl group such as a monocyclic, bicyclic or tricyclic aryl group or R₈ and R₉ may combine and form a saturated or unsaturated fused 5- or 6-membered ring which may be substituted.

R₁₀, R₁₁, R₁₂ and R₁₃ each represents a hydrogen atom or an alkyl, i.e. an unsubstituted or substituted alkyl group such as a straight-chain, branched chain or cyclic alkyl group and moreover, R₁₀, R₁₁, R₁₂ and R₁₃ represents an aryl, i.e., an unsubstituted or substituted aryl group such as a monocyclic, bicyclic or tricyclic aryl group.

Further, any two of R₁₀, R₁₁, R₁₂ and R₁₃ may combine and form an unsubstituted or substituted 5- or 6-membered ring. Preferred are carbocyclic rings.

R₁₄, R₁₅, R₁₆ and R₁₇ each represents a hydrogen atom or an alkyl group, i.e., an unsubstituted or substituted alkyl group such as a straight-chain, branched chain or cyclic alkyl group and moreover, R₁₄, R₁₅, R₁₆ and R₁₇ each represents an aryl, i.e., an unsubstituted or substituted aryl group such as a monocyclic or bicyclic aryl group.

Further, R₁₄, R₁₅, R₁₆ and R₁₇ each represents an unsubstituted or substituted alkoxy group, for example, an alkoxyl group where the alkyl moiety thereof is a straight-chain or branched chain alkyl moiety; an unsubstituted or substituted aryloxy group, for example, an aryloxy group where the aryl moiety thereof is monocyclic or bicyclic; an unsubstituted or substituted acyl group, for example, an alkylacyl group where the alkyl moiety thereof is a straight-chain or branched chain alkyl moiety or an arylacyl group where the aryl moiety thereof is monocyclic or bicyclic; an unsubstituted or substituted alkoxycarbonyl group, for example, an alkoxycarbonyl group where the alkyl moiety thereof is a straight-chain or branched chain alkyl moiety; an unsubstituted or substituted benzoyl group; an unsubstituted or substituted ureido group, for example, an alkylureido group where the alkyl moiety thereof is a straight-chain or branched chain alkyl moiety or an arylureido group where the aryl moiety thereof is monocyclic or bicyclic; an unsubstituted or substituted amino group, for example, a mono- or di-alkylamino group where the alkyl moiety thereof is a straight-chain or branched chain alkyl moiety or a mono- or di-arylamino group where the aryl moiety thereof is a monocyclic or bicyclic; an unsubstituted or substituted amido group, for example, a mono- or di-alkylamido group where the alkyl moiety thereof is a straight-chain or branched chain alkyl moiety or a mono- or di-arylamido group where the aryl moiety thereof is monocyclic or bicyclic; an unsubstituted or substituted sulfamido group, for example, an alkylsulfamido group where the alkyl moiety thereof is a straight chain or branched chain alkyl moiety or an arylsulfamido group where the aryl moiety thereof is monocyclic or bicyclic; an unsubstituted or substituted carbamyl group, for example, an alkylcarbamyl group where the alkyl moiety thereof is a straight chain or branched chain alkyl moiety or an arylcarbamyl group where the aryl moiety thereof is monocyclic or bicyclic; an unsubstituted or substituted sulfamoyl group, for example, an alkylsulfamoyl group where the alkyl moiety thereof is a straight chain or branched chain alkyl moiety or an arylsulfamoyl group where the aryl moiety thereof is monocyclic or bicyclic; a halogen atom such as a bromine atom, a chlorine atom, an iodine atom or a fluorine atom; a nitro group; a cyano group; a hydroxy group; or a carboxy group, or any adjacent two of R₁₄ to R₁₇ may combine and form a saturated or unsaturated 5- or 6-membered ring which may have other rings fused therewith.

Q represents a pharmaceutically acceptable anion necessary for electrical charge balance, j and k each is 1 or 2 and m and n each is 0 or 1.

More specifically, as described above, R₁ and R₃ individually can represent an alkyl group which may be unsubstituted or substituted. Suitable examples of alkyl groups include straight-chain, branched chain and cyclic alkyl groups having 1 to 15 carbon atoms, more preferably 1 to 10 carton atoms, even more preferably 1 to 8 carbon atoms.

Specific examples of alkyl groups for R₁ and R₃ include methyl, ethyl, n-propyl, i-propyl, n-butyl, i-butyl, sec-butyl, tert-butyl, n-pentyl, hexyl, heptyl, octyl, cyclopropyl, cyclopentyl, cyclohexyl, 2-propenyl, 2-butenyl, 3-hexenyl and the like. Specific examples of suitable substituents which can be present on the alkyl group when R1 and R3 represent a substituted alkyl group include halogen atoms such as chlorine, bromine, fluorine and iodine, an aryl group, an alkoxy group, a hydroxy group, and the like. A preferred number of carbon atoms for the unsubstituted and substituted alkyl groups for R1 and R3 ranges from 1 to 15, more preferably 1 to 10, most preferably 1 to 8. Specific examples of alkyl groups substituted by halogen atoms include trifluoromethyl, trifluoroethyl, tetrafluoropropy and pentafluoropropyl group.

As defined above, R₂, R₆ and R₇ represents an alkyl group which can be a straight-chain, branched chain or cyclic alkyl group and which may be substituted. Suitable examples of alkyl groups and substituents thereon are as described above for R₁ and R₃. A preferred number of carbon atoms for the alkyl group represented by R₂, R₆ and R₇ is from 1 to 15 carbon atoms, more preferably 1 to 10 carbon atoms, most preferable is 1 to 8 carbon atoms.

The aryl group represented by R₂, R₆ and R₇ above can be a monocyclic, bicyclic or tricyclic aryl group such as a phenyl group, a biphenyl group, a naphthyl group or an anthracenyl group and such may be unsubstituted or substituted. Suitable examples of substituents which can be present on the aryl group represented by R₂, R₆ and R₇ include one or more of a halogen atom such as chlorine, bromine, fluorine or iodine, an alkyl group, an alkoxy group, a hydroxy group, a nitro group, a cyano group, an amino group, an alkyl- or aryl-substituted amino group, an acylamino group, a sulfonylamino group, a carbamoyl group, a sulfamoyl group, a carboxyl group, an alkoxycarbonyl group, and the like. A suitable number of carbon atoms for the aryl group for R₂, R₆ and R₇ is 6 to 20, preferably 6 to 15, more preferably 6 to 8.

The heterocyclic ring represented by R₂, R₆ and R₇ can be a 5- to 6-membered heterocyclic ring containing one or more oxygen atoms, sulfur atoms or nitrogen atoms as hereto atoms. Suitable examples of heterocyclic rings represented by R₂, R₆ and R₇ include an imidazole ring, a thiazole ring, a pyrrole ring, a pyrazole ring, a furan ring, a thiophene ring, a piperidine ring, a morpholine ring, a piperadine ring, a pyrazine ring, a pyridine ring, a pyrimidine ring, and the like. These heterocyclic rings may be substituted, for example, by substituents as described above for the aryl group for R₂, R₆ and R₇ or may be condensed with another ring such as a saturated or unsaturated ring.

Examples of alkyl groups represented by R₄ and R₅ include unsubstituted or substituted alkyl groups having from 1 to 15 carbon atoms, more preferably 1 to 10 carbon atoms. Suitable examples of suitable alkyl groups include those described above for R₁ and R₃ and substituents which can be present on the alkyl group represented by R₄ and R₅ include an alkyl group, an alkoxy group, a hydroxy group, a cyano group, a halogen atom, and the like.

Examples of alkyl groups represented by R₆ and R₇ above include alkyl groups as described above for R₄ and R₅. A suitable number of carbon atoms for the alkyl group for R₆ and R₇ is 1 to 15 carbon atoms, more preferably 1 to 10 carbon atoms. Further, R₆ and R₇ represents an unsubstituted or substituted aryl group which includes monocyclic, bicyclic and tricyclic aryl groups. A suitable number of carbon atoms for the aryl group for R₆ and R₇ is 6 to 20 carbon atoms, more preferably 6 to 15 carbon atoms. Specific examples of suitable aryl groups for R₆ and R₇ and substituents therefore include those described above for R2.

The alkyl group represented by R₈, R₉, R₁₀, R₁₁, R₁₂, R₁₃, R₁₄, R₁₅, R₁₆ and R₁₇ above can be straight-chain, branched chain or cyclic and can include 1 to 15 carbon atoms, more preferably 1 to 10 carbon atoms, even more preferably 1 to 8 carbon atoms. The alkyl group represented by R₈, R₉, R₁₀, R₁₁, R₁₂, R₁₃, R₁₄, R₁₅, R₁₆ and R₁₇ can also be a unsubstituted alkyl group. Specific examples of alkyl group for R₈, R₉, R₁₀, R₁₁, R₁₂, R₁₃, R₁₄, R₁₅, R₁₆ and R₁₇ include methyl, ethyl, n-propyl, i-propyl, 2-propenyl, n-butyl, i-butyl, sec-butyl, tert-butyl, n-pentyl, hexyl, heptyl, octyl, cyclopropyl, cyclopentyl, cyclohexyl and the like. Specific examples of suitable substituents which can be present on the alkyl group when R₈, R₉, R₁₀, R₁₁, R₁₂, R₁₃, R₁₄, R₁₅, R₁₆ and R₁₇ represent a substituted alkyl group include halogen atoms such as chlorine, bromine, fluorine and iodine, an aryl group, an alkoxy group, a hydroxy group, and the like. A preferred number of carbon atoms for the unsubstituted and substituted alkyl groups for R₈, R₉, R₁₀, R₁₁, R₁₂, R₁₃, R₁₄, R₁₅, R₁₆ and R₁₇ ranges from 1 to 15, more preferably 1 to 10.

The aryl group represented by R₈, R₉, R₁₀, R₁₁, R₁₂, R₁₃, R₁₄, R₁₅, R₁₆ and R₁₇ can be a monocyclic, bicyclic or tricyclic aryl group such as a phenyl group, a biphenyl group, a naphthyl group or an anthracenyl group and such may be unsubstituted or substituted. Suitable examples of substituents which can be present on the aryl group represented by R₈-R₁₇ include one or more of a halogen atom such as chlorine, bromine, fluorine or iodine, an alkyl group, an alkoxy group, a hydroxy group, a nitro group, a cyano group, an amino group, an alkyl- or aryl-substituted amino group, an acylamino group, a sulfonylamino group, a carbamoyl group, a sulfamoyl group, a carboxyl group, an alkoxycarbonyl group, and the like. A suitable number of carbon atoms for the aryl group for R₈ to R₁₇ is 6 to 20, preferably 6 to 15.

Examples of the rings formed by binding R₈ with R₉ includes a benzene ring, naphthalene ring, dihydronaphthalene ring, anthracene ring, and phenanthrene ring. Suitable examples of substituents which can further be present on the rings formed by binding R₈ with R₉ include halogen atoms, a hydroxy group, an alkyl group preferably having 1 to 5 carbon atoms, an aryl group preferably having 6 to 8 carbon atoms, an alkoxy group preferably having 1 to 5 carbon atoms, an aryloxy group preferably having 6 to 8 carbon atoms, an alkoxycarbonyl group preferably having 2 to 6 carbon atoms and acyloxy group preferably having 2 to 6 carbon atoms. More preferable substituents are one or more chlorine atoms, one or more fluorine atoms, a methoxy group, an ethoxy group, a trifluoromethyl group, a methoxycarbonyl group, a phenyl group and a methylenedioxy group.

Moreover, two of R₁₀, R₁₁, R₁₂ and R₁₃ may combine and form a 5- or 6-membered carbocyclic ring. A suitable number of carbon atoms for the carbocyclic ring including substituent groups thereon or R₁₀, R₁₁, R₁₂ and R₁₃ is 3 to 15 carbon atoms, preferably 3 to 10 carbon atoms.

Typical examples of 5- and 6-membered carbocyclic rings include a cyclopentane ring, a cyclopentene ring, a cyclohexane ring, a cyclohexene ring and the like.

Z₁ and Z₂, which may be the same or different, each represents an atomic group necessary to form a saturated or unsaturated 5- and 6-membered ring. Moreover, the ring formed by Z₁ and Z₂ can be substituted with one or more substituents or can be condensed with another ring such as a saturated or unsaturated ring, e.g., a cyclohexene ring, a benzene ring or a naphthalene ring. Suitable examples of substituents which can be present on the ring formed by Z₁ and Z₂ include one or more of an alkyl group, an alkoxy group, an aryloxy group, a halogen atom (such as chlorine, bromine, fluorine and iodine), an aryl group, a hydroxy group, an amino group, an alkyl- or aryl-substituted amino group, an acylamino group, a sulfonylamino group, a carbamoyl group, a sulfamoyl group, a carboxyl group, an alkoxycarbonyl group, an acyloxy group, a heterocyclic ring (such as a pyrrole ring, a furan ring, a piperidine ring, a morpholine ring, a pyridine ring, etc.) a cyano group, a nitro group, and the like, and suitable examples of saturated or unsaturated rings condensed therewith include a cyclopentene ring, a cyclohexene ring, a benzene ring, a naphthalene ring, an anthracene ring, a phenanthrene ring, a thiophene ring, a pyridine ring, etc.

Specific examples of heterocyclic rings formed by Z₁ and Z₂ include 5- and 6-membered heterocyclic rings such as those including nuclei comprising those of the thiazole series (e.g., thiazole, 4-methylthiazole, 4-phenylthiazole, 4,5-diphenylthiazole, 4,5-dimethylthiazole, etc.), those of the benzothiazole series (e.g., benzothiazole, 5-chlorobenzothiazole, 5-methylbenzothiazole, 5-phenylbenzothiazole, 5-methoxybenzothiazole, 4-fluorobenzothiazole, 5,6-dioxymethylenebenzothiazole, 5-nitrobenzothiazole, 5-trifluoromethylbenzothiazole, 5-methoxycarbonylbenzothiazole, 5-hydroxybenzothiazole, 6-hydroxybenzothiazole, 5-cyanobenzothiazole, 5-iodobenzothiazole, etc.), those of the naphthothiazole series (e.g., .alpha.-naphthothiazole, .beta.-naphthothiazole, .gamma.-naphthothiazole, 5-methoxy-.beta.-naphthothiazole, 8-methoxy-.alpha.-naphthothiazole, 6-methoxy-8-acetyloxy-.beta.-naphthothiazole, 8,9-dihydro-. beta.-naphthothiazole, etc.), those of the oxazole series (e.g., 4-methyloxazole, 4,5-diphenyloxazole, 4-phenoxyoxazole, etc.), those of the benzoxazole series (e.g., benzoxazole, 5-chlorobenzoxazole, 5,6-dimethylbenzoxazole, 6-hydroxybenzoxazole, 5-phenylbenzoxazole, etc.), those of the naphthoxazole series (e.g., .alpha.-naphthoxazole, .beta.-naphthoxazole, etc.), those of the selenazole series (e.g., 4-methylselenazole, 4-phenylselenazole, etc.), those of the benzoselenazole series (e.g., benzoselenazole, 5-chlorobenzoselenazole, 5-methoxybenzoselenazole, 5-hydroxybenzoselenazole, etc.), those of the thiazoline series (e.g., thiazoline, 4,4-dimethylthiazole, etc.), those of the 2-pyridine series (e.g., 2-pyridine, 5-methyl-2-pyridine, 5-methoxy-2-pyridine, 4-chloro-2-pyridine, 5-carbamoyl-2-pyridine, 5-methoxycarbonyl-2-pyridine, 4-acetylamino-2-pyridine, 6-methylthio-2-pyridine, 6-methyl-2-pyridine etc.), those of the 4-pyridine series (e.g., 4-pyridine, 3-methoxy-4-pyridine, 3,5-dimethyl-4-pyridine, 3-chloro-4-pyridine, 3-methyl-4-pyridine, etc.), those of the 2-quinoline series (e.g., 2-quinoline, 6-methyl-2-quinoline, 6-chloro-2-quinoline, 6-ethoxy-2-quinoline, 6-hydroxy-2-quinoline, 6-nitro-2-quinoline, 6-acetylamino-2-quinoline, 6-dimethylaminocarbonyl-2-quinoline, 8-fluoro-2-quinoline, etc.), those of the 4-quinoline series (e.g., 4-quinoline, 6-methoxy-4-quinoline, 6-acetylamino-4-quinoline, 8-chloro-4-quinoline, 6-trifluoromethyl-4-quinoline, etc.), those of the 1-isoquinoline series (e.g., 1-isoquinoline, 6-methoxy-1-isoquinoline, 6-chloro-1-isoquinoline, etc.), those of the 3,3-dialkylindolenine series (e.g., 3,3-dimethylindolenine, 3,3,7-trimethylindolenine, 5-chloro-3,3-dimethylindolenine, 5-ethoxycarbonyl-3,3-dimethylindolenine, 5-nitro-3,3-dimethylindolenine, 3,3-dimethyl-4,5-phenyleneindolenine, 3,3-dimethyl-6,7-phenyleneindolenine, 5-acetylamino-3,3-dimethylindolenine, 5-diethylamino-3,3-dimethylindolenine, 5-methanesulfonylamino-3,3-dimethylindolenine, 5-benzoylamino-3,3-dimethylindolenine, etc.), those of the imidazole series (e.g., imidazole, 1-alkyl-4-phenylimidazole, 1-alkyl-4,5-dimethylimidazole, etc.), those of the benzimidazole series (e.g., benzimidazole, 1-alkylbenzimidazole, 1-alkyl-5-trifluorobenzimidazole, 1-alkyl-5-chlorobenzimidazole, 1-alkyl-5-sulfamoylbenzimidazole, 1-aryl-5-methoxycarbonylbenzimidazole, 1-alkyl-5-acetylaminobenzimidazole, 1-alkyl-5-nitrobenzimidazole, 1-alkyl-5-diethylaminobenzimidazole, 1-alkyl-5-pentyloxybenzimidazole, etc.), those of naphthimidazole series (e.g., 1-alkyl-.alpha.-naphthimidazole, 1-alkyl-5-methoxy-.beta.-naphthimidazole, etc.) and like rings.

Suitable examples of substituents which can be present on the L, L₂ or L₃ substituted methine group include an alkyl group (e.g., methyl, ethyl, butyl, etc.), an aryl group (e.g., phenyl, tolyl, etc.), a halogen atom (e.g., chlorine, bromine, fluorine and iodine), or an alkoxy group (e.g., methoxy, ethoxy, etc.) and suitable rings formed by the combination of L₁ and R₃ include a 5-membered heterocyclic ring (e.g., a pyrroline ring, etc.) and a 6-membered heterocyclic ring (e.g., a tetrahydropyridine ring, an oxazine ring, etc.).

The term “pharmaceutically acceptable anion” for Q which is necessary for electrical charge balance in the compounds above is intended to mean an ion, when administered to a subject in need thereof, is non-toxic and renders the compounds above soluble in aqueous systems.

Suitable examples of pharmaceutically acceptable anions represented by Q include halides such as chloride, bromide and iodide, sulfonates such as aliphatic and aromatic sulfonates, e.g., methanesulfonate, trifluoromethanesulfonate, p-toluenesulfonate, naphthalenesulfonate, 2-hydroxyethanesulfonate, and the like, sulfamates such as cyclohexanesulfamate, sulfates such as methyl sulfate and ethyl sulfate, bisulfates, borates, alkyl and dialkyl phosphates such as diethyl phosphate and methylhydrogen phosphate, pyrophosphates such as trimethylpyrophosphate and diethyl hydrogen pyrophosphate, carboxylates, advantageously carboxy- and hydroxy-substituted carboxylates and carbonates. Preferred examples of pharmaceutically acceptable anions include chloride, bromide, iodide, acetate, propionate, valerate, citrate, maleate, fumarate, lactate, succinate, tartrate and benzoate.

In particular, rhodacyanine compounds of the Formulae (I) to (V) where Y1 is a sulfur atom, L₂ and L₃ are unsubstituted methine group and anion represented by Q is chloride, bromide, iodide, p-toluenesulfonate or acetate are preferred.

Non-limiting examples of DLC compounds of general Formulae (I) to (V), the cation moiety of which has log P value of 4.5-12, which can be employed in this invention, include the compounds 1-96 depicted in FIGS. 8a-o.

According to one embodiment of the invention, the DLC compound used according to the method of this aspect of the invention is MKT-077 [also known as FJ-776; 1-ethyl-2-{[3-ethyl-5-(3-methylbenzothiazolin-2-yliden]-4-oxothiazolidin-2-ylidenemethyl}pyridium chloride].

Additional DLC compounds which can be used according to this aspect of the invention include those described in Kawakami M., et al., 1998, “Structure-activity of novel rhodacyanine dyes as antitumor agents”, J. Med. Chem. 41: 130-142; and Kawakami M., et al., 1998, “A self-sensitized photoreaction of rhoacyanine dye, MKT 077”, Tetrahedron Letters 39: 1763-1766; Takasu K., et al., 2004, Bioorganic and Medical Chemistry Letters, 14: 1689-1692; Muthyala R., et al., 2001, Photochemistry and Photobiology 74: 837-845; all of which are incorporated herein by reference.

The DLC compounds of the invention can be easily produced from known starting materials in accordance with the methods disclosed in British Patent Nos. 489,335 and 487,051; in U.S. Pat. Nos. 2,388,963, 2,454,629, 2,504,468, 2,536,986 and 2,961,318, the disclosure of which is incorporated herein by reference.

It will be appreciated that in order to increase to solubility of the DLC compounds of the invention (e.g., for use by intravenous administration), the DLC compounds can be prepared with a cyclodextrin as such α-cyclodextrin, β-cyclodextrin, γ-cyclodextrin which are prepared by reacting starch or dextrin with amylase.

DLC compounds which can be used in accordance with the teachings of the invention can be identified using methods well known in the art which are further described in Example 3 of the Examples section which follows. For example, in vitro assays which employ cells expressing mortalin can be used to identify DLC compounds which are capable of down-regulating mortalin function [e.g., the binding of mortalin to protein members of the complement MAC complex, such as the C9 protein (GenBank Accession No. NP_{—}001728.1; SEQ ID NO:9), mortalin-mediated anti-senescence activity, mortalin-mediated p53 sequestration and mortalin-mediated antigen presentation]. The ability of the DLC compound to bind to mortalin and optionally inhibit mortalin and/or the binding of mortalin to members of the MAC complex can be determined using well-known in vitro assays such as those described in Example 3 of the Examples section which follows. Candidate compounds can be further qualified by in vitro assays for their effect on mortalin-mediated, complement-mediated cell lysis (see Example 3 of the Examples section which follows).

As mentioned hereinabove and exemplified in the Examples section which follows, MKT-077 significantly enhances complement-mediated cell lysis when used together with specific antibodies (anti-cancer antibodies). Thus, according to some embodiments of this aspect of the invention anti-cancer antibody therapy is administered to the subject, prior to, concomitant with or following administration of the DLC compound. One of ordinary skill in the art will be able to determine the preferred protocol of administration such as based on the turn-over of the administered compounds.

As used herein the phrase “anti-cancer antibody therapy” refers to the treatment of cancer which is effected using an antibody directed against a constitutively or transiently expressed surface epitope which is predominantly expressed on cells of the cancer/tumor.

As used herein, the term “antibody” refers to an intact antibody molecule or a fragment thereof.

As used herein, the phrase “antibody fragment” refers to a functional fragment of an antibody that is capable of binding to an epitope of an antigen and directly or indirectly recruits the complement system.

Antibody fragments include complementarity-determining region (CDR) of an immunoglobulin light chain (referred to herein as “light chain”), a CDR of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single-chain Fv, an Fab, an Fab′, an F(ab′)2 and a single domain molecule such as VH and VL.

Generally, antibody fragments have the advantage of generally being smaller than a whole antibody while retaining essentially a substantially identical binding specificity of the antibody comprising the immunoglobulin variable regions of the antibody fragment. Thus, antibody fragments are less likely to include moieties capable of causing steric hindrance inhibiting binding of the antibody to the antigen and have superior biodistribution and diffusion properties (for example, systemically in-vivo, or in isolated tissues) than the latter.

When an immune response is desired, e.g., for activation of the complement, the antibody fragment of the invention includes a functional constant region or a portion thereof such as an Fc region, which may be conveniently attached to a functional moiety (e.g., a toxic moiety).

Depending on the application and purpose, any of the various isotypes of a constant region or a portion thereof may be employed. For example, for inducing antibody-dependent cell mediated cytotoxicity (ADCC) by a natural killer (NK) cell, the isotype can be IgG; for inducing ADCC by a mast cell/basophil, the isotype can be IgE; and for inducing ADCC by an eosinophil, the isotype can be IgE or IgA. For inducing a complement cascade the antibody or antibody fragment can include a constant region or portion thereof capable of initiating the cascade. For example, the antibody or antibody fragment may advantageously comprise a Cgamma2 domain of IgG or Cmu3 domain of IgM to trigger a C1q-mediated complement cascade.

Methods of generating monoclonal and polyclonal antibodies are well known in the art. Antibodies may be generated via any one of several known methods, which may employ induction of in vivo production of antibody molecules, screening of immunoglobulin libraries (Orlandi, R. et al. (1989). Cloning immunoglobulin variable domains for expression by the polymerase chain reaction. Proc Natl Acad Sci USA 86, 3833-3837; and Winter, G. and Milstein, C. (1991). Man-made antibodies. Nature 349, 293-299), or generation of monoclonal antibody molecules by continuous cell lines in culture. These include, but are not limited to, the hybridoma technique, the human B-cell hybridoma technique, and the Epstein-Barr virus (EBV)-hybridoma technique (Kohler, G. and Milstein, C. (1975). Continuous cultures of fused cells secreting antibody of predefined specificity. Nature 256, 495-497; Kozbor, D. et al. (1985). Specific immunoglobulin production and enhanced tumorigenicity following ascites growth of human hybridomas. J Immunol Methods 81, 31-42; Cote R J. et al. (1983). Generation of human monoclonal antibodies reactive with cellular antigens. Proc Natl Acad Sci USA 80, 2026-2030; and Cole, S. P. et al. (1984). Human monoclonal antibodies. Mol Cell Biol 62, 109-120).

In cases where target antigens are too small to elicit an adequate immunogenic response when generating antibodies in vivo, such antigens (referred to as “haptens”) can be coupled to antigenically irrelevant carriers such as keyhole limpet hemocyanin (KLH) or serum albumin (e.g., bovine serum albumin (BSA)) carriers (see, for example, U.S. Pat. Nos. 5,189,178 and 5,239,078). Coupling a hapten to a carrier can be effected using methods well known in the art. For example, direct coupling to amino groups can be effected and optionally followed by reduction of the imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill., USA. The resulting immunogenic complex can then be injected into suitable mammalian subjects such as mice, rabbits, and others. Suitable protocols involve repeated injection of the immunogen in the presence of adjuvant according to a schedule designed to boost production of antibodies in the serum. The titers of the immune serum can readily be measured using immunoassay procedures which are well known in the art.

The antisera obtained can be used directly or monoclonal antibodies may be obtained, as described hereinabove.

It will be appreciated that for human therapy or diagnostics, humanized antibodies can be used. Humanized forms of non-human (e.g., murine) antibodies are genetically engineered chimeric antibodies or antibody fragments having e.g., minimal portions derived from non-human antibodies. Humanized antibodies include antibodies in which the CDRs of a human antibody (recipient antibody) are replaced by residues from a CDR of a non-human species (donor antibody), such as mouse, rat, or rabbit, having the desired functionality. In some instances, the Fv framework residues of the human antibody are replaced by corresponding non-human residues. Humanized antibodies may also comprise residues found neither in the recipient antibody nor in the imported CDR or framework sequences. In general, the humanized antibody will comprise substantially all of at least one, and typically two, variable domains, in which all or substantially all of the CDRs correspond to those of a non-human antibody and all or substantially all of the framework regions correspond to those of a relevant human consensus sequence. Humanized antibodies optimally also include at least a portion of an antibody constant region, such as an Fc region, typically derived from a human antibody (see, for example: Jones, P. T. et al. (1986). Replacing the complementarity-determining regions in a human antibody with those from a mouse. Nature 321, 522-525; Riechmann, L. et al. (1988). Reshaping human antibodies for therapy. Nature 332, 323-327; Presta, L. G. (1992b). Curr Opin Struct Biol 2, 593-596; and Presta, L. G. (1992a). Antibody engineering. Curr Opin Biotechnol 3(4), 394-398).

Methods for humanizing non-human antibodies are well known in the art. Generally, a humanized antibody has one or more amino acid residues introduced into it from a source which is non-human. These non-human amino acid residues are often referred to as imported residues, which are typically taken from an imported variable domain. Humanization can be performed essentially as described (see, for example: Jones et al. (1986); Riechmann et al. (1988); Verhoeyen, M. et al. (1988). Reshaping human antibodies: grafting an antilysozyme activity. Science 239, 1534-1536; and U.S. Pat. No. 4,816,567), by substituting human CDRs with corresponding rodent CDRs. Accordingly, humanized antibodies are chimeric antibodies, wherein substantially less than an intact human variable domain has been substituted by the corresponding sequence from a non-human species. In practice, humanized antibodies may be typically human antibodies in which some CDR residues and possibly some framework residues are substituted by residues from analogous sites in rodent antibodies.

Human antibodies can also be produced using various additional techniques known in the art, including phage-display libraries (Hoogenboom, H. R. and Winter, G. (1991). By-passing immunization. Human antibodies from synthetic repertoires of germline VH gene segments rearranged in vitro. J Mol Biol 227, 381-388; Marks, J. D. et al. (1991). By-passing immunization. Human antibodies from V-gene libraries displayed on phage. J Mol Biol 222, 581-597; Cole et al. (1985), Monoclonal Antibodies and Tumoral disease Therapy, Alan R. Liss, Inc., pp. 77-96; and Boerner, P. et al. (1991). Production of antigen-specific human monoclonal antibodies from in vitro-primed human splenocytes. J Immunol 147, 86-95). Humanized antibodies can also be created by introducing sequences encoding human immunoglobulin loci into transgenic animals, e.g., into mice in which the endogenous immunoglobulin genes have been partially or completely inactivated. Upon antigenic challenge, human antibody production is observed in such animals which closely resembles that seen in humans in all respects, including gene rearrangement, chain assembly, and antibody repertoire. Ample guidance for practicing such an approach is provided in the literature of the art (for example, refer to: U.S. Pat. Nos. 5,545,807; 5,545,806; 5,569,825; 5,625,126; 5,633,425; and 5,661,016; Marks, J. D. et al. (1992). By-passing immunization: building high affinity human antibodies by chain shuffling. Biotechnology (N.Y.) 10(7), 779-783; Lonberg et al., 1994. Nature 368:856-859; Morrison, S. L. (1994). News and View: Success in Specification. Nature 368, 812-813; Fishwild, D. M. et al. (1996). High-avidity human IgG kappa monoclonal antibodies from a novel strain of minilocus transgenic mice. Nat Biotechnol 14, 845-851; Neuberger, M. (1996). Generating high-avidity human Mabs in mice. Nat Biotechnol 14, 826; and Lonberg, N. and Huszar, D. (1995). Human antibodies from transgenic mice. Int Rev Immunol 13, 65-93).

After antibodies have been obtained, they may be tested for activity, for example via enzyme-linked immunosorbent assay (ELISA).

As mentioned the anti-cancer antibody of the invention specifically binds to an antigen of the tumor cell-of-interest (e.g., by recognizing a membrane bound tumor specific antigen).

Examples of such membrane bound tumor specific antigen are well known in the art and include but are not limited to markers such as CD20 which is expressed on B cells in non-Hodgkin lymphoma, human epidermal growth factor 2 (HER2) which is expressed on breast cancer cells, CD33 which is expressed on hematopoietic stem cells in acute myelogenous leukemia (AML), CD52 which is expressed on B-cells in chronic lymphocytic leukemia (CLL), epidermal growth factor receptor (EGFR) which is expressed on cancerous cells in colorectal cancer disease and head & neck cancer diseases, and insulin-like growth factor-I receptor (IGF-IR) which is expressed in various cancerous tumors such as breast cancer (e.g., which is associated with BRCA1 mutations), non-small cell lung cancer, head and neck cancer, neuroblastoma and prostate cancer. Other targets may be identified using gene expression analysis (e.g., array technology) and antibodies directed thereto may be generated according to the above teachings.

A non-limiting list of such antibodies or targets are listed in Table 1 below.

TABLE 1
Anti-cancer antibodies

| | | | | Approved |
|---|---|---|---|---|
| MAb Name | Trade Name | Used to Treat: | target | in: |
| Rituximab | RITUXAN | Non-Hodgkin lymphoma | CD20 | 1997 |
| Trastuzumab | HERCEPTIN | Breast cancer disease | HER2 | 1998 |
| Gemtuzumab | MYLOTARG | Acute myelogenous | CD33 | 2000 |
| ozogamicin* | | leukemia (AML) | | |
| Alemtuzumab | CAMPATH | Chronic lymphocytic | CD52 | 2001 |
| | | leukemia (CLL) | | |
| Ibritumomab tiuxetan* | ZEVALIN | Non-Hodgkin lymphoma | CD20 | 2002 |
| Tositumomab* | BEXXAR | Non-Hodgkin lymphoma | CD20 | 2003 |
| Cetuximab | ERBITUX | Colorectal cancer disease | EGFR | 2004 |
| | | Head & neck cancer | | 2006 |
| | | diseases | | |
| CP 751, 871 | | various cancers and | IGF-IR | |
| | | rheumatoid arthritis | | |
| Panitumumab | | Metastatic colorectal | EGFR | |
| | | cancer | | |

Types of cancer or tumoral diseases which can be treated via the method of the invention include benign tumors, warts, polyps, precancers, and malignant tumors/cancer.

The cancer which can be treated according to the method of this aspect of the invention can be any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

Precancers are well characterized and known in the art (refer, for example, to Berman J J. and Henson D E., 2003. Classifying the precancers: a metadata approach. BMC Med Inform Decis Mak. 3:8). Classes of precancers amenable to treatment via the method of the invention include acquired small or microscopic precancers, acquired large lesions with nuclear atypia, precursor lesions occurring with inherited hyperplastic syndromes that progress to cancer, and acquired diffuse hyperplasias and diffuse metaplasias. Examples of small or microscopic precancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia). Examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Examples of precursor lesions occurring with inherited hyperplastic syndromes that progress to cancer include atypical mole syndrome, C cell adenomatosis and MEA. Examples of acquired diffuse hyperplasias and diffuse metaplasias include AIDS, atypical lymphoid hyperplasia, Paget's disease of bone, post-transplant lymphoproliferative disease and ulcerative colitis.

The cancer which can be treated according to the method of this aspect of the invention can be characterized by cancerous cells expressing mortalin (e.g., as set forth by SEQ ID NO:1 for the amino acid sequence) and SEQ ID NO:2 for the nucleic acid sequence) and/or complement regulatory proteins (mCRPs) [e.g., the decay accelerating factor (DAF, CD55, GenBank Accession No. NP_{—}000565.1; SEQ ID NO:3), membrane cofactor protein (MCP, CD46, e.g., GenBank Accession No. NP_{—}002380.3, SEQ ID NO:4, or NM_{—}002389.3, SEQ ID NO:5) and CD59 (e.g., GenBank Accession NO. NP_{—}000602.1, SEQ ID NO:6; NM_{—}000611.4, SEQ ID NO:7)].

It will be appreciated that the DLC compound and/or the anti-cancer antibody of the invention can be administered to an organism per se, or in a pharmaceutical composition where it is mixed with suitable carriers or excipients.

As used herein a “pharmaceutical composition” refers to a preparation of one or more of the active ingredients described herein (i.e., the DLC compound of the invention) with other chemical components such as physiologically suitable carriers and excipients. The purpose of the pharmaceutical composition is to facilitate administration of the active ingredients to the subject.

Hereinafter, the phrases “physiologically acceptable carrier” and “pharmaceutically acceptable carrier” which may be interchangeably used refer to a carrier or a diluent that does not cause significant irritation to the subject and does not abrogate the biological activity and properties of the administered active ingredients. An adjuvant is included under these phrases.

Herein, the term “excipient” refers to an inert substance added to the pharmaceutical composition to further facilitate administration of an active ingredient of the invention. Examples, without limitation, of excipients include calcium carbonate, calcium phosphate, various sugars and types of starch, cellulose derivatives, gelatin, vegetable oils and polyethylene glycols. The pharmaceutical composition may advantageously take the form of a foam or a gel.

Techniques for formulation and administration of drugs may be found in “Remington's Pharmaceutical Sciences,” Mack Publishing Co., Easton, Pa., latest edition, which is incorporated herein by reference.

Suitable routes of administration include any of various suitable systemic and/or local routes of administration.

Suitable routes of administration may, for example, include the inhalation, oral, buccal, rectal, transmucosal, topical, transdermal, intradermal, transnasal, intestinal and/or parenteral routes; the intramuscular, subcutaneous and/or intramedullary injection routes; the intrathecal, direct intraventricular, intravenous, intraperitoneal, intranasal, and/or intraocular injection routes; and/or the route of direct injection into a tissue region of the subject.

The pharmaceutical composition may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

For transmucosal administration, penetrants appropriate to the barrier to be permeated are used in the formulation. Such penetrants are generally known in the art.

For oral administration, the pharmaceutical composition can be formulated readily by combining the active ingredients with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration via the inhalation route, the active ingredients for use according to the invention can be delivered in the form of an aerosol/spray presentation from a pressurized pack or a nebulizer with the use of a suitable propellant, e.g., a fluorochlorohydrocarbon such as dichlorodifluoromethane, trichlorofluoromethane, dichloro-tetrafluoroethane; carbon dioxide; or a volatile hydrocarbon such as butane, propane, isobutane, or mixtures thereof. In the case of a pressurized aerosol, the dosage unit may be determined by providing a valve to deliver a metered amount. Capsules and cartridges of, e.g., gelatin for use in a dispenser may be formulated containing a powder mix of the active ingredients and a suitable powder base such as lactose or starch.

The pharmaceutical composition may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

A pharmaceutical composition for parenteral administration may include an aqueous solution of the active ingredients in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredients may be in powder form for constitution with a suitable vehicle, e.g., sterile, pyrogen-free water based solution, before use.

The pharmaceutical composition may also be formulated in rectal compositions such as suppositories or retention enemas, using, e.g., conventional suppository bases such as cocoa butter or other glycerides.

According to one embodiment of the invention the pharmaceutical composition which comprises the DLC compound of the invention (e.g., MKT-077) is formulated for intravenous administration.

The pharmaceutical composition should contain the active ingredients in an amount effective to achieve disease treatment.

As mentioned hereinabove, the DLC compound of the invention (e.g., the cationic rhodacyanine compound such as MKT-077) can be administered prior to, concomitantly with, or following administration of the antibody therapy.

According to one embodiment of the invention administration of the DLC compound is effected prior to administration of the anti-cancer antibody. Thus, the DLC compound (e.g., MKT-077) can be administered about 1-4 days prior to administration of the anti-cancer antibody, about 24 hours prior to administration of the anti-cancer antibody, or alternatively, about 1-20 hours, 1-10 hours or 1-5 hours prior to administration of the anti-cancer antibody.

For example, the DLC compound can be injected intravenously at non-toxic doses prior (1, 2 or 4 hours) to injection of the antibodies. A bolus weekly combined treatment of the DLC compound (e.g., the rhodacyanine compound) followed by antibody treatment can be applied for 4 weeks with a 6 weeks rest and this regimen may be repeated for 2, 3 or more cycles. Alternatively, the DLC compound of the invention can be administered continuously by using a micropump for up to 7-10 days before antibody treatment.

According to another embodiment of the invention, the DLC compound (e.g., MKT-077) is administered concomitantly with the administration of the anti-cancer antibody. It will be appreciated that concomitant administration can be effected using a single or several administrations of the active agents (i.e., the DLC compound and the anti-cancer antibody of the invention).

It will be appreciated that administration of the DLC compound is effected at doses sufficiently low to avoid toxicity yet sufficient to serve as adjuvant for the antibody therapy. Animal models can be used for formulating the dose and regimen. Xenograft tumor mouse models are well established. Following orthotopic inoculation of human tumor cells or xenografts into immunodeficient nude or SCID/beige mice, the mice develop primary tumors at the site of injection and also develop distant metastases in the liver or lung. Therefore, these models perfectly mimic human cancer development. Alternatively, models of mouse tumor cells injected into syngeneic immunocompetent naive mice are available. For example, EL4 T lymphoma cells in C57Bl/6 mice. Anti-cancer antibodies can be injected intravenously into tumor-bearing mice to examine the optimal therapeutic capacity of this model of anti-cancer antibody therapy. By using such tumor models, the IC₅₀ of the DLC compound of the invention in the absence and presence of antibody therapy can be studied.

Thus, determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Calculations of dosage in mass per kg, for manufactured drugs and supplements, can be made on the basis of conversion of animal doses to human equivalent doses (HED), based on body surface area, as published by the FDA [Guidance for industry and reviewers, estimating the safe starting dose in clinical trials for therapeutics in adult healthy volunteers; website: http://www.fda.gov/cber/guidelines.htm]. See Table 2 below.

TABLE 2

| | To convert animal | To convert animal dose | |
|---|---|---|---|
| | dose in mg/kg to | in mg/kg to HED^{a} in | |
| | dose in mg/m², | mg/kg, either: | |
| | multiply by the km^{d} | Divide animal | Multiply Animal |
| Species | below: | dose by: | dose by: |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel | 7 | 5.3 | 0.19 |
| monkey | | | |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, human equivalent dose (HED) can be calculated from the formula: HED = animal dose in mg/kg × (animal weight in kg/human weight in kg) 0.33;
^{b}This km is provided for reference only since healthy children will rarely be volunteers for phase 1 trials;
^{c}For example, cynomolgus, rhesus, stumptail;
^{d}The km (for mass constant) is not truly constant for any species, but increases within a species as body weight increases.

The following describes exemplary doses and treatment regimens of the DLC compound of the invention for mammals:

Mice: 5-10 mg/kg bolus intravenous (i.v.) daily X5, 2 days intervals

Rats: 1-5 or 1-3 mg/kg bolus i.v. daily X5

Dogs: 0.1-0.4 mg/kg bolus i.v. daily X5 days

Dosage amount and interval may be adjusted individually to provide tissue levels of the active ingredients which are sufficient to achieve the desired therapeutic effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of the composition to be administered will be dependent on the subject being treated, the severity of the affliction, the manner of administration, the judgment of the prescribing physician, etc.

As mentioned, when used in cancer patients by continuous i.v. infusion at doses of 150 mg/m²/five days (i.e., 30 mg/m², day, for 5 days) MKT-077 administration was associated with renal toxicity (Propper D J., et al., Annals Onc. 10: 923-927, 1999). Thus, for example, it is suggested to use MKT077 at a concentration with is sufficiently low to avoid accumulative renal damage.

Thus, according to one embodiment of the invention, when administered as a continuous intravenous infusion, MKT-077 can be administered for 1-5 days at a dosage in the range of about 0.1-25 mg/m²/day. For example, it is suggested to use MKT-077 at a concentration of about 0.1-25, 0.1-20, 0.1-15, 0.1-10, 0.1-5, 0.1-1, 1-25, 1-20, 1-15, 1-10, 1-5 or 10-20 mg/m²/day, for continuous i.v. infusion of about 1-10, 1-8, 1-6, 1-5, 4, 3, 2 days or 1 day.

According to Britten C D., et al., Clin. Cancer Res. 6: 42-49, 2000, a bolus dose (30 minutes i.v. infusion) of 126 mg/m²/week X 4 weeks every 6 weeks was well tolerated but were associated with hypomagnesemia (kidney toxicity). This was shown to result in MKT-077 plasma concentrations (1.2-6.3 microgram/ml) that are higher than the IC₅₀ concentrations required in vitro for human tumor cells tested (0.15-0.5 microgram/ml).

Thus, the invention contemplates bolus dosing in the range of about 1-126 mg/m²/week, for example in the range of about 0.1-100, 1-100, 10-100, 20-100, 40-100, 50-100 mg/m² MKT-077 once a week (bolus, 30 minutes i.v.) for 4 weeks.

For example, for combined therapy of lymphoma such as diffuse Large B-Cell NHL, in which RITUXAN is provided at a dose of 375 mg/m² IV per infusion given on day 1 of each cycle of chemotherapy for up to 8 infusions, the DLC compound of the invention (e.g., MKT-077) can be provided at a bolus dose (e.g., 30 minutes i.v.) of about 50-100 mg/m² 4-7 days prior to RITUXAN administration or alternatively at a continuous infusion of about 10-20 mg/m²/day for 4-5 days prior to RITUXAN administration.

For example, for combined therapy of patients with metastatic breast cancer whose tumors over-express the HER2 protein, the DLC compound of the invention (e.g., MKT-077) can be provided at a bolus dose (30 minutes i.v.) of about 50-100 mg/m² 4-7 days prior to the administration of 4 mg/kg Trastuzumab by intravenous (IV) infusion over 90 minutes. Alternatively, the DLC compound of the invention (e.g., MKT-077) can be provided at a continuous infusion of about 10-20 mg/m²/day for 4-5 days prior to the administration of 4 mg/kg Trastuzumab by intravenous (IV) infusion over 90 minutes.

In addition, for the combined therapy of acute myeloid leukemia (AML), in which Gemtuzumab Ozogamicin is administered by I.V. at a dose of 9 mg/m² for a total of 2 doses, given on days 0 and 14, the DLC compound of the invention (e.g., MKT-077) can be provided at a bolus dose (30 minutes i.v.) of about 50-100 mg/m² 4-7 days prior to the administration of Gemtuzumab Ozogamicin.

In addition, the invention further contemplates a unit dosage form comprising MKT-077 in a range of about 0.17-220 mg. For example, the unit dosage form of the invention comprises about 0.17 mg MKT-077, about 1.7 mg MKT-077, about 17 mg MKT-077, about 34 mg MKT-077, about 0.17-42 mg MKT-077, about 5-10 mg MKT-077, about 5-15 mg MKT-077, about 10-20 mg MKT-077, about 10-40 mg MKT-077, about 10-100 mg MKT-077, about 50-100 mg MKT-077, about 75-125 mg MKT-077, about 100-150 mg MKT-077, about 100-200 mg MKT-077 or about 150-214 mg MKT-077.

Compositions of the invention may, if desired, be presented in a pack or dispenser device, such as an FDA approved kit, which may contain one or more unit dosage forms containing the active ingredients. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

The DLC compound of the invention (e.g., the cationic rhodacyanine compound such as MKT-077) can be suitably formulated as pharmaceutical compositions which can be suitably packaged as an article of manufacture. Such an article of manufacture comprises a packaging material which comprises a label for use as an adjuvant anti-cancer therapy, wherein the packaging material packaging the delocalized lipophilic cation (DLC) compound of the invention.

The article of manufacture can further include the anti-cancer antibody therapy described hereinabove.

It will be appreciated that the DLC compound and the anti-cancer antibody of the invention can be packaged in separate containers, in a single container, or can be co-formulated.

The packaging material can be identified in print in or on the packaging material for treatment of cancer (e.g., tumoral disease).

As used herein the term “about” refers to ±10%.

Additional objects, advantages, and novel features of the invention will become apparent to one ordinarily skilled in the art upon examination of the following examples, which are not intended to be limiting. Additionally, each of the various embodiments and aspects of the invention as delineated hereinabove and as claimed in the claims section below finds experimental support in the following examples.

### Examples

Reference is now made to the following examples, which together with the above descriptions, illustrate the invention in a non limiting fashion.

Generally, the nomenclature used herein and the laboratory procedures utilized in the invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

Cell culture and cell lysates—K562, a human erythroleukemic cell line, was cultured in RPMI-1640 supplemented with 10% (v/v) heat-inactivated fetal bovine serum (GIBCO laboratories, Grand Island, N.Y., USA), 1% glutamine, 2% pyruvate and antibiotics mixture (Bio-Lab, Jerusalem, Israel). The HCT116 colon carcinoma cell line and its p53 knockout derivative were a gift from Prof. Moshe Oren (Department of Molecular Cell Biology and Immunology, The Weizmann Institute of Science, Rehovot, Israel) and were maintained in McCoy's medium supplemented with 10% fetal bovine serum and antibiotics. All cell lines were grown at 37° C. in a humidified atmosphere of 5% CO₂ in air. To prepare cell lysates, 40×10⁶ cells were mixed with 1 ml lysis buffer composed of 100 mM Tris, pH 7.5, 10 mM EDTA, protease inhibitor cocktail (Sigma, Rehovot, Israel) and 0.7% Triton-X100. After three cycles of freezing and thawing, the cell lysate was subjected to centrifugation for 15 minutes at 14 000 g and the supernatant was collected and diluted with 1 ml HBSS (Sigma).

Sera, antisera and reagents—Normal human serum (NHS) was prepared from healthy individuals. Heat inactivation of NHS was performed by incubation at 56° C. for 30 minutes. Human sera were kept frozen at −70° C. in small aliquots and thawed only once. Purified human C9 protein was purchased from Advanced Research Technologies (San Diego, Calif., USA). A polyclonal antiserum directed to K562 cells was prepared in rabbits. Mouse anti-mortalin antibodies were purchased from Stressgen (Ann Arbor, Mich., USA). Peroxidase-conjugated goat anti-mouse IgG and Peroxidase-conjugated Rabbit anti-Goat IgG were purchased from Jackson Immunoresearch (West Grove, Pa., USA). Streptolysin O (SLO), A23187, Rhodamine123 and BSA were purchased from Sigma. MKT-077 was kindly provided by Keizo Koya (Synta Pharmaceuticals Corp. 45 Hartwell Avenue, Lexington, Mass. 02421 USA).

Cell lysis and collection of secreted vesicles—Cytotoxicity assay was performed as described before [Reiter, Y. and Z. Fishelson, Complement membrane attack complexes induce in human leukemic cells rapid expression of large proteins (L-CIP). Mol Immunol, 1992. 29(6): p. 771-81]. Briefly, for a cell lysis assay, cells were incubated with diluted anti-K562 antiserum for 30 minutes at 4° C. and then with complement [NHS or heat-inactivated normal human serum (HI-NHS), 50%] for 60 minutes at 37° C. Cell lysis was determined by Trypan blue inclusion. Statistical significance was analyzed by using the two-sided unpaired Student's t-test. For collection of secreted vesicles, cells were treated with antibodies for 30 minutes at 4° C. and then with NHS or HI-NHS (50%) for 10 minutes at 37° C. Then, they were extensively washed in HBSS and incubated at 37° C. After another 10 minutes, cells were removed by centrifugation at 250 g and supernatants were sedimented first at 5000 g to remove cell debris. Then they were subjected to centrifugation at 100,000 g, conditions known to spin down small membrane vesicles.

Protein analysis—Protein concentration was analyzed with the BCA Protein Assay Kit (Pierce, Rockford, Ill., USA). Proteins were subjected to SDS-PAGE under reducing conditions [50 mM dithiothreitol (DTT)], in a 10% acrylamide gel and then transferred onto a nitrocellulose membrane (Schleicher & Schuell, Dassel, Germany). The membrane was blocked with 5% skim milk (Tnuva, Rehovot, Israel) in Tris-buffered saline containing 0.05% Tween 20 (TBST) for 1 hour at room temperature. The membrane was treated with rabbit anti-mortalin antibodies or goat anti-C9 antibodies and then with peroxidase-conjugated goat anti-rabbit IgG or peroxidase-conjugated rabbit anti-goat IgG. Bands were developed with an enhanced chemiluminescence reagent (Pierce) and exposed to a SuperRX film (Fuji, Tokyo, Japan).

Binding of mortalin to complement C9—Binding of mortalin to the complement C9 protein was examined by ELISA analysis. In brief, purified human C9 and BSA (0.5 μg per well each) were bound overnight at 4° C. to a 96-well Enzyme Immunoassay (EIA) plate (Corning, N.Y., USA). Diluted cell lysate prepared from 40×10⁶ K562 cells as described above was added to each well for 60 minutes at 37° C. After washes, monoclonal mouse anti-mortalin antibody (diluted 1:500) was added for 30 minutes at 37° C. and then peroxidase-conjugated goat anti-mouse IgG was added for 30 minutes at room temperature. Next, o-phenylenediamine (Sigma) was added and the plate was examined in a Spectrafluor Plus plate reader (Tecan, Austria) at 450 nm.

### Example 1

### The Mortalin Inhibitor, MKT-077, is Capable of Sensitizing Cells to Complement-Mediated Cell Lysis

Mortalin is involved in the defense of cells against complement-mediated lysis (Pilzer and Fishelson, Int. Immunol. 17: 1239-1248, 2005). Since MKT-077 is a known inhibitor of mortalin, the present inventors have examined the effect of MKT-077 on complement-mediated cell lysis, as follows.

Experimental Results

MKT-077 sensitizes cells to complement-mediated lysis—Human K562 erythroleukemia cells were pretreated for 60 minutes at 37° C. with MKT-077, at different concentrations, and then the cells were treated for additional 60 minutes at 37° C. with anti-K562 antibodies for 30 minutes on ice and with complement (NHS) or heat-inactivated NHS (HI-NHS) as control. Finally, cells were washed and percent cell lysis was quantified by Trypan Blue inclusion. The results presented in FIG. 1 show that MKT-077 significantly enhances killing of K562 cells by complement in the presence of specific antibodies.

MKT-077 prevents release of mortalin and C9 by vesiculation after complement attack—Mortalin plays a major role in the removal of the complement membrane attack complex (MAC), including C9, by vesiculation from cells attacked by complement (Pilzer and Fishelson, Int. Immunol. 17: 1239-1248, 2005). The present inventors have tested whether or not MKT-077 treatment prevents the vesiculation of mortalin and C9, as follows. K562 cells were pretreated for 1 hour with 200 μM or 50 μM MKT-077 and then treated for 10 minutes with sublytic doses of anti-K562 antibodies and NHS or HI-NHS. Cells were washed 4 times with Hank's Balanced Salt Solution (HBSS) and then incubated again for 10 minutes at 37° C. Cells were sedimented and supernatant were collected. The supernatant were subjected to centrifugation first at 5,000 g to remove cell debris and then at 100,000 g to spin down small membrane vesicles. The high-speed pellets and supernatant were subjected to SDS-PAGE and Western Blotting. Analysis with anti-mortalin antibodies (FIG. 2a) and anti-C9 antibodies (FIG. 2b) indicated that the release of both mortalin and C9 by vesiculation was inhibited by the treatment with MKT-077.

The effect of MKT-077 on complement-mediated lysis is p53-independent—MKT-077 is known to prevent the binding of mortalin to p53 (Wadhwa et al. Cancer Res. 15: 6818-6821, 2000). To determine if the observed effect of MKT-077 on complement-mediated lysis is p53-dependent, the present inventors have studied HCT p53 +/+ and HCT p53 −/− cells. Both cells were pretreated with MKT-077 and then treated with anti-K562 antibodies and NHS or HI-NHS. After 60 minutes at 37° C., percent lysis was measured. The results showed that the presence or absence of p53 had no effect on the complement sensitization effect of MKT-077 (FIG. 3).

MKT-077 has no effect on SLO-mediated lysis and A23187-mediated lysis—The present inventors have further addressed the question of specificity of the effect of MKT-077 on cell death. The effect of MKT-077 on cell death induced by Streptolysin O (SLO), another cytolytic pore-forming protein, was examined. Cells were pretreated with MKT-077 and then treated with several doses of SLO or with dithiothreitol (DTT) as control. Lysis was measured after 15 minutes incubation at 37° C. Results presented in FIG. 4a show no effect of MKT-077 on SLO-mediated lysis. The present inventors have further examined the effect of MKT-077 on cell death induced by the Ca⁺² ionophore A23187. Cells were treated with MKT-077 and then with several doses of A23187 or dimethyl sulfoxide (DMSO) as control. Next, the cells were washed and percent cell death was measured. The results presented in FIG. 4b showed no effect of MKT-077 on A23187-mediated lysis.

MKT-077 interferes with mortalin binding to C9—Mortalin binds to C9 (Pilzer and Fishelson, Int. Immunol. 17: 1239-1248, 2005) and MKT-077 binds to mortalin. To test whether binding of MKT-077 to mortalin affects the binding of mortalin to C9, C9 and bovine serum albumin (BSA) were bound to wells of a microtiter plate, incubated with K562 lysates (source for mortalin), with or without MKT-077, and then treated with anti-mortalin antibodies followed by incubation with HRP-conjugated secondary antibodies and OPD (o-phenylenediamine) colorimetry (analyzed in an ELISA reader). Results presented in FIG. 5 indicate a significant decrease in the ability of mortalin to bind C9 in the presence of MKT-077.

The effect of MKT-077 on cell death induced by complement is specific—Rhodamine123 is a cationic rhodacyanine dye similar to MKT-077. To test if other rhodacyanine molecules can cause a similar effect on complement-mediated lysis as MKT-077 the following experiment was performed. Lysis of K562 cells upon treatment with antibodies and NHS or HI-NHS was tested after pretreatment with either MKT-077 or rhodamine123. As shown in FIG. 6, Rhodamine123, unlike MKT-077 which is capable of inhibiting the binding of mortalin to C9, had no effect on complement-mediated lysis. Hence, the sensitization effect on cells lysed by complement is specific to DLC compounds such as MKT-077 which are capable of inhibiting mortalin or the binding of mortalin to MAC complex components such as C9. In addition, the effect of MKT-077 on complement-mediated lysis was found to be mortalin-dependent (data not shown).

Altogether, these results demonstrate that MKT-077 can be used to sensitize cells to complement mediated cell lysis in a mortalin-depended manner and suggest its use as an adjuvant for anti-cancer antibody therapy.

### Example 2

### In Vivo Experiments Using MKT-077 as an Adjuvant for Anti-Cancer Antibody Therapy

Raji human B lymphoma and K562 human erythroleukemia cells (3×10⁶ cells) are injected subcutaneously into CD1 athymic nude mice. 10-14 days following injection, tumor mass of 5-10 mm diameter develops under the skin of the injected mice. MKT-077 (at a dosage of: 5-10 mg/kg bolus i.v. daily X5, 2 days intervals) is injected intravenously and 1-2 hours later the mice receive an intravenous injection of the specific anti-cancer antibodies, as follows. Raji bearing mice receive Rituximab (MabThera, Roche Pharmaceuticals, Israel, a chimeric humanized anti-CD20 antibody) at a dosage of 375 mg/m² (bolus or continuous i.v.) and K562 tumor bearing mice receive rabbit anti-K562 antibodies at a dosage of 1 mg total IgG/mouse (bolus or continuous i.v.).

### Example 3

### Identification of DLC Compounds as Adjuvant for Anti-Cancer Antibody Therapy

In order to identify additional DLC compounds which can be used as adjuvant for anti-cancer antibody therapy, the present inventors have developed several screening assays which test the suitability of candidate DLC compounds as adjuvant, as follows.

Identification of DLC compounds capable of inhibiting the binding of mortalin to protein members of the MAC complex (e.g., C9): As described in Example 1, hereinabove, MKT-077 binds to mortalin and interferes with its binding to complement C9 (FIG. 5). In order to identify additional DLC compounds which can be used as adjuvant for anti-cancer antibody therapy, the present inventors have developed a high throughput ELISA (enzyme-linked immunosorbent assay) in which purified C9 (which can be obtained from Complement Technology, Tyler, Tex., USA; SEQ ID NO:9; NM_{—}001737) is attached to a microtiter plate and mortalin (SEQ ID NO:1) is added either alone or mixed with a potential DLC inhibitor such as MKT-077. The amount of C9-bound mortalin is then examined with rabbit anti-mortalin antibody (prepared by the present inventors) or mouse anti-mortalin antibody (purchased from Stressgen, Ann Arbor, Mich., USA) and a second peroxidase-labeled antibody.

Identification of DLC compounds which are capable of specifically binding mortalin: An ELISA assay in which purified mortalin (SEQ ID NO:1) is attached to ELISA plates and labeled candidate DLC molecules are added the plates. Detection is performed using a Plate Reader (e.g. Tecan, Austria) and measuring color intensity or fluorescence using suitable wavelength for excitation and emission.

Identification of DLC compounds which are capable of specifically binding mortalin-expressing cells: Cells expressing mortalin and cells not expressing mortalin (e.g., cells in which mortalin expression is downregulated) are contacted with a plurality of DLC compounds under conditions suitable for cell-compound complex formation. The formation of the cell-compound complex can be monitored using well known immunological assays such as ELISA, FACS analysis, Western blotting, immunohistochemistry and/or radioimmuno assay. The compounds (e.g., MKT-077) which are capable of forming a cell-compound complex with the cells expressing mortalin but not with the cells when not-expressing mortalin can be further tested (as described hereinbelow) for suitability of being used as adjuvant for anti-cancer antibody therapy.

Qualification of candidate DLC compounds for use as adjuvant for anti-cancer antibody therapy: The inhibitory effect of a candidate DLC compound (e.g., an MKT-077-like reagent) on complement-induced mortalin release and complement-induced lysis is tested with K562 human erythroleukemia cells treated with a sublytic dose of rabbit anti-K562 cells antibody and complement (normal human serum). Supernatant is collected and the amount of released mortalin and complement C9 is assessed by Western blotting (as shown in FIG. 2a) and the viability of the cells is determined by Trypan blue inclusion (as shown in FIG. 1) (cell cytotoxicity assay).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the invention.

### REFERENCES

### (Additional References are Provided in Text)

1. Britten C D., et al., 2000, Clin. Cancer Res. 6: 42-49;
2. Fishelson Z. et al. Mol. Immunol. 40: 109-123, 2003;
3. Donin N. et al. Clin. Exp. Immunol. 131: 254-263, 2003;
4. Kawakami M., et al., 1998, “Structure-activity of novel rhodacyanine dyes as antitumor agents”, J. Med. Chem. 41: 130-142;
5. Kawakami M., et al., 1998, “A self-sensitized photoreaction of rhoacyanine dye, MKT 077”, Tetrahedron Letters 39: 1763-1766;
6. Kraus S and Fishelson Z, 2000, Eur. J. Immunol. 30:1272-1280
7. Pilzer D and Fishelson Z, 2005, Int. Immunol. 17: 1239-1248
8. Propper D. J. et al., 1999. Ann. Oncol., 10: 923-927
9. Wadhwa, R., et al., Cancer Res, 2000. 60: 6818-21
10. U.S. Pat. No. 5,670,530
11. U.S. patent application Ser. No. 11/440,132

1. A method of treating cancer in a subject in need thereof, the subject being treated with anti-cancer antibody therapy, the method comprising administering to the subject a therapeutically effective amount of a delocalized lipophilic cation (DLC) compound capable of binding mortalin, thereby treating the cancer in the subject.

2-4. (canceled)

5. A pharmaceutical composition comprising as an active ingredient a delocalized lipophilic cation (DLC) compound and an anti-cancer antibody, said delocalized lipophilic cation (DLC) being capable of binding mortalin, and a pharmaceutically acceptable carrier.

6. The method of claim 1, wherein said administering said delocalized lipophilic cation (DLC) is effected prior to administration of the anti-cancer antibody therapy.

7. The method of claim 1, wherein said administering said delocalized lipophilic cation (DLC) is effected concomitant with said administration of the anti-cancer antibody therapy.

8-10. (canceled)

11. The method of claim 1, wherein said delocalized lipophilic cation (DLC) is a cationic rhodacyanine compound.

12. The method of claim 11, wherein said cationic rhodacyanine compound is MKT-077.

13. The method of claim 1, wherein said anti-cancer antibody is selected from the group consisting of Rituximab, Trastuzumab, Gemtuzumab, ozogamicin, Alemtuzumab, Ibritumomab tiuxetan, Tositumomab, Cetuximab, Bevacizumab, CP-751,871 and Panitumumab.

14. The method of claim 1, wherein the anti-cancer antibody therapy is directed against a tumor associated antigen selected from the group consisting of CD20, HER2, CD33, CD52, EGFR and IGF1R.

15. The method of claim 12, wherein said MKT-077 is formulated for intravenous administration.

16. The method of claim 15, wherein administration of said MKT-077 is effected at a dosage of 0.1-25 mg/m²/day.

17. The method of claim 15, wherein administration of said MKT-077 is effected at a dosage of 1-126 mg/m²/week.

18. The method of claim 1, wherein the anti-cancer antibody therapy is RITUXAN and whereas administration of said anti-cancer antibody is effected at a dosage of 375 mg/m².

19. A method of identifying a delocalized lipophilic cation (DLC) adjuvant for anti-cancer antibody therapy, comprising:
(a) contacting cells expressing mortalin with a plurality of delocalized lipophilic cation (DLC) compounds, and;
(b) identifying at least one compound from said plurality of compounds which is capable of down-regulating a mortalin function, said at least one compound being the adjuvant for anti-cancer antibody therapy.

20. The method of claim 19, wherein said mortalin function comprising binding to a complement protein.

21. The method of claim 20, wherein said complement protein is a C9 protein as set forth by SEQ ID NO:9.

22. A unit dosage form comprising 0.17-220 milligram of MKT-077.

23. The pharmaceutical composition of claim 5, wherein said delocalized lipophilic cation (DLC) is a cationic rhodacyanine compound.

24. The pharmaceutical composition of claim 23, wherein said cationic rhodacyanine compound is MKT-077.

25. The pharmaceutical composition of claim 5, wherein said anti-cancer antibody is selected from the group consisting of Rituximab, Trastuzumab, Gemtuzumab, ozogamicin, Alemtuzumab, Ibritumomab tiuxetan, Tositumomab, Cetuximab, Bevacizumab, CP-751,871 and Panitumumab.

26. The pharmaceutical composition of claim 5, wherein said anti-cancer antibody is directed against a tumor associated antigen selected from the group consisting of CD20, HER2, CD33, CD52, EGFR and IGF1R.

27. The pharmaceutical composition of claim 24, wherein said MKT-077 is formulated for intravenous administration.
